(19) United States
(12) Patent Application Publication
(10) Pub. No.: US 2015/0111919 A1
(43) Pub. Date: Apr. 23, 2015

(54) COMPOSITIONS AND METHODS FOR TREATING MULTIPLE SCLEROSIS

(71) Applicant: Biovista, Inc., Charlottesville, VA (US)

(72) Inventors: Spyros Deftereos, Athens (GR); Andreas Persidis, Athens (GR)

(21) Appl. No.: 14/251,818

(22) Filed: Apr. 14, 2014

Related U.S. Application Data
(63) Continuation of application No. 13/123,660, filed on Apr. 11, 2011, now abandoned.

(51) Int. Cl. A61K 31/444

(52) U.S. Cl. CPC A61K 31/444
USPC 514/292

(57) ABSTRACT

Described herein are compositions and methods for treating multiple sclerosis. In particular, described herein are compositions that include one or more dimebolins and/or pharmaceutically acceptable salts thereof and methods for using the compositions for treating multiple sclerosis.

### CROSS REFERENCE TO RELATED APPLICATIONS

This application claims priority under 35 USC §119(e) to U.S. Provisional Application Ser. No. 61/104,854, filed on Oct. 13, 2008, and U.S. Provisional Application Ser. No. 61/157,687 filed on Mar. 5, 2009, the entire disclosure of each of which is incorporated herein by reference.

### TECHNICAL FIELD

The invention described herein relates to methods for treating multiple sclerosis. In particular, the invention relates to methods for treating multiple sclerosis by administering therapeutically effective amounts of one or more dimebolins, and/or pharmaceutically acceptable salts thereof.

### BACKGROUND AND SUMMARY OF THE INVENTION

Multiple sclerosis (MS) is an inflammatory demyelinating disease of the central nervous system (CNS). MS may take several forms, with new symptoms occurring either in discrete attacks (relapsing forms) or slowly accumulating over time (progressive forms). Between attacks, though symptoms may disappear completely, often permanent neurological problems yet occur, especially as the disease advances. Even so, symptoms of MS usually appear in episodic acute periods that may worsen upon recurrence (relapses, exacerbations, bouts, and/or attacks). With such recurrence, the condition may develop into a gradually-progressive deterioration of neurologic function. Many subtypes of MS present in a combination of both acute episodes and gradual deterioration. The most common presentation of MS is the clinically isolated syndrome (CIS), in which a patient has an attack suggestive of demyelination, but does not fulfill the criteria for multiple sclerosis. It has been reported that only 30 to 70% of persons experiencing CIS later develop MS.

Several specific subtypes, or patterns of progression of the disease, have been described. The United States National Multiple Sclerosis Society has standardized four subtype definitions as relapsing remitting, secondary progressive, primary progressive, and progressive relapsing. Those subtypes are characterized by using the past course of the disease as one means of predicting the future course. It is appreciated that the accurate characterization of the subtype may be useful in both the prognosis of the disease and also in therapeutic decisions.

The relapsing-remitting subtype is characterized by unpredictable relapses which may be followed by periods of relative quiet (remission) with no new signs of disease activity. Such periods of relative quiet may last months or even years. The deficits suffered during attacks may either resolve or leave sequelae. The relapsing-remitting subtype is the most common subtype and describes the initial course of 85-90% of individuals with MS. When deficits always resolve between attacks, this subtype may also be referred to as benign MS.

The secondary progressive MS subtype also includes initial relapsing-remitting MS, but where the afflicted patient then begins to have progressive neurological decline between acute attacks without any definite periods of remission. However, occasional relapses and minor remissions may appear. The median time between disease onset and conversion from relapsing-remitting to secondary progressive MS has been reported to be 19 years.

The primary progressive subtype describes the approximately 10-15% of individuals who never have remission after their initial MS symptoms. The subtype is further characterized by progression of disability from onset, with no, or only occasional and minor, remissions and improvements. Generally, the age of onset for the primary progressive subtype is later than other subtypes.

The progressive relapsing MS subtype is characterized by those individuals who, from onset, have a steady neurological decline but also suffer clear superimposed attacks. This subtype is the least common of all subtypes.

In addition to the standard subtypes, cases with non-standard behavior have also been described. Those non-standard subtypes are sometimes referred to as borderline forms of multiple sclerosis, and include Devic's disease, Balo concentric sclerosis, Schilder's diffuse sclerosis, and Marburg multiple sclerosis. However, it has been alternately reported that those non-standard subtypes may either be atypical variants of MS or instead different diseases.

MS is a major cause of disability, because in most patients the disease ultimately has a progressive course. In most patients, the progressive course of the disease manifests itself during or after a preceding phase of relapses and remissions (secondary progressive disease), whereas in a small percentage of patients (10-15%) the disease course is progressive from onset (primary progressive disease). Most currently available treatments for multiple sclerosis are aimed at suppressing the inflammatory component of the disease. Their main clinical impact is on relapses whereas an effect on permanent disability is less well established. Patients with primary progressive MS show less inflammatory activity, which is one of the reasons why they are frequently excluded from treatment trials, despite clear clinical progression. Recent evidence suggests that axonal loss may occur earlier in the disease course of MS than previously anticipated. Further, such early axonal loss may be the pathologic correlate of irreversible disability. MS is also frequently characterized by plaques or lesions of demyelination in the nerve fibers of the brain and spinal cord. Demyelination causes multiple and varied neurological symptoms and signs, usually with relapses and exacerbations.

The clinical course of MS is highly variable and unpredictable, with many patients experiencing acute episodes of exacerbations, followed by periods of remission. The disease progresses at various paces to a chronic, degenerative condition. Frequently, a diagnosis of MS may not be made for many years after the onset of symptoms because the symptoms can be variable, sporadic, and similar to those associated with other disorders. As the disease progresses, patients are frequently unable to remain fully ambulatory, and their functional systems steadily decline. The most severe cases of MS are characterized by paralysis or even death.

The precise causes of MS are not yet known, though several theories have been proposed. Research to date has indicated that the etiology of MS may in fact be related to a combination of factors, such as autoimmunity, environmental, viral and genetic factors. Thus, there remains a need to identify additional treatments for MS which can treat the disease, minimize the effects of the disease, and/or slow the progression of the disease.

It has been discovered that dimebolins, and pharmaceutically acceptable salts thereof, are useful in treating patients suffering from or in need of relief from multiple sclerosis. The use of dimebolins, or pharmaceutically acceptable salts thereof, in treating multiple sclerosis has heretofore been unknown.

In one embodiment of the invention described herein, methods are described for treating MS. In another embodiment, methods are described herein for treating primary progressive MS. In another embodiment, methods are described herein for treating secondary progressive MS. In another embodiment, methods are described herein for treating relapsing remitting MS. In another embodiment, methods are described herein for treating progressive relapsing MS. In another embodiment, the methods described herein are useful for treating either progressive form of MS, including primary progressive MS and/or secondary progressive MS. In one aspect, the methods described herein include the step of administering a therapeutically effective amount of one or more dimebolins and/or pharmaceutically acceptable salts thereof to a patient suffering from, or in need of relief from one or more forms of multiple sclerosis, or borderline forms of multiple sclerosis.

In another embodiment, the method includes the step of administering a therapeutically effective amount of one or more dimebolins and/or pharmaceutically acceptable salts thereof, and administering a therapeutically effective amount of one or more NMDA receptor antagonists to a patient suffering from, or in need of relief from one or more forms of multiple sclerosis, or borderline forms of multiple sclerosis. In another embodiment, the method includes the step of administering a therapeutically effective amount of one or more dimebolins and/or pharmaceutically acceptable salts thereof, and administering a therapeutically effective amount of one or more HMG-CoA reductase inhibitors, also referred to as statins, to a patient suffering from, or in need of relief from one or more forms of multiple sclerosis, or borderline forms of multiple sclerosis. In another embodiment, the method includes the step of administering a therapeutically effective amount of one or more dimebolins and/or pharmaceutically acceptable salts thereof, and administering a therapeutically effective amount of one or more immunosuppressive drugs to a patient suffering from, or in need of relief from one or more forms of multiple sclerosis, or borderline forms of multiple sclerosis. In another embodiment, the method includes the step of administering a therapeutically effective amount of one or more dimebolins and/or pharmaceutically acceptable salts thereof, and administering a therapeutically effective amount of one or more immunomodulatory drugs to a patient suffering from, or in need of relief from one or more forms of multiple sclerosis, or borderline forms of multiple sclerosis. In another embodiment, the method includes the step of administering a therapeutically effective amount of one or more dimebolins and/or pharmaceutically acceptable salts thereof, administering a therapeutically effective amount of one or more NMDA receptor antagonists, and administering a therapeutically effective amount of one or more immunosupressive drugs to a patient suffering from, or in need of relief from one or more forms of multiple sclerosis, or borderline forms of multiple sclerosis. In another embodiment, the method includes the step of administering a therapeutically effective amount of one or more dimebolins and/or pharmaceutically acceptable salts thereof, administering a therapeutically effective amount of one or more NMDA receptor antagonists, and administering a therapeutically effective amount of one or more immunomodulatory drugs to a patient suffering from, or in need of relief from one or more forms of multiple sclerosis, or borderline forms of multiple sclerosis.

In another embodiment, compositions useful for treating MS are described herein. In one embodiment, the compositions are useful for treating any form, or combination of forms of MS, including primary progressive MS, secondary progressive MS, relapsing remitting MS, and/or progressive relapsing MS. In another embodiment, the compositions described herein are useful for treating either progressive form of MS, including primary progressive MS and/or secondary progressive MS. In one aspect, the compositions described herein include a therapeutically effective amount of one or more dimebolins and/or pharmaceutically acceptable salts thereof to a patient suffering from, or in need of relief from one or more forms of multiple sclerosis, or borderline forms of multiple sclerosis.

In another embodiment, the compositions include a therapeutically effective amount of one or more dimebolins and/or pharmaceutically acceptable salts thereof, and a therapeutically effective amount of one or more NMDA receptor antagonists. In another embodiment, the compositions include a therapeutically effective amount of one or more dimebolins and/or pharmaceutically acceptable salts thereof, and a therapeutically effective amount of one or more HMG-CoA reductase inhibitors. In another embodiment, the compositions include a therapeutically effective amount of one or more dimebolins and/or pharmaceutically acceptable salts thereof, and a therapeutically effective amount of one or more immunosuppressive drugs. In another embodiment, the compositions include a therapeutically effective amount of one or more dimebolins and/or pharmaceutically acceptable salts thereof, and a therapeutically effective amount of one or more immunomodulatory drugs. In another embodiment, the compositions include a therapeutically effective amount of one or more dimebolins and/or pharmaceutically acceptable salts thereof, a therapeutically effective amount of one or more NMDA receptor antagonists, and a therapeutically effective amount of one or more immunosuppressive drugs. In another embodiment, the compositions include a therapeutically effective amount of one or more dimebolins and/or pharmaceutically acceptable salts thereof, a therapeutically effective amount of one or more NMDA receptor antagonists, and a therapeutically effective amount of one or more immunomodulatory drugs.

### BRIEF DESCRIPTION OF THE DRAWING

FIG. 1 shows the average clinical score for treatment groups 3F (1 mg/kg ip, ▴) and 5F (10 mg/kg ip, ▪), compared to saline vehicle treated control (∘) and dexamethasone (1 mg/kg ip, positive control, ♦) over a 35 day observation period of an acute EAE murine model of MS.

### DETAILED DESCRIPTION

Described herein are new methods for treating MS by administering a therapeutically effective amount of one or more dimebolins, or pharmaceutically acceptable salts thereof. Also described herein are compositions for treating MS, where the compositions include a therapeutically effective amount of one or more dimebolins, or pharmaceutically acceptable salts thereof. In one embodiment, the methods include the step of administering a therapeutically effective amount of one or more dimebolins, or pharmaceutically acceptable salts thereof to a patient suffering from or in need of relief from MS.

In another illustrative embodiment, pharmaceutical compositions are described herein. Illustrative pharmaceutical compositions include dosage forms of one or more dimebolins and/or pharmaceutically acceptable salts thereof, and one or more pharmaceutically acceptable carriers, excipients, and/or diluents therefor. Other illustrative pharmaceutical compositions include (a) mixtures of one or more dimebolins and/or pharmaceutically acceptable salts thereof, and one or more NMDA antagonists, (b) mixtures of one or more dimebolins and/or pharmaceutically acceptable salts thereof, and one or more immunosuppressive drugs, (c) mixtures of one or more dimebolins and/or pharmaceutically acceptable salts thereof, and one or more immunomodulatory drugs, (d) mixtures of one or more dimebolins and/or pharmaceutically acceptable salts thereof, one or more NMDA antagonists, and one or more immunosupressive drugs, and (e) mixtures of one or more dimebolins and/or pharmaceutically acceptable salts thereof, one or more NMDA antagonists, and one or more immunomodulatory drugs. Other illustrative formulations include “sandwich” formulations where two or more separate drug dosage forms are conveniently adhered one to the other for simultaneous co-administration.

As used herein, the term “dimebolin” generally refers to hydrogenated pyrido[4,3-b]indoles, such as the compounds described herein, and pharmaceutically acceptable salts of the foregoing. It is also to be understood that in each of the foregoing, any corresponding pharmaceutically acceptable salt is also included in the illustrative embodiments described herein. Illustrative derivatives include, but are not limited to, both those compounds that may be synthetically prepared from the compounds described herein, as well as those compounds that may be prepared in a similar way as those described herein, but differing in the selection of starting materials. One such dimebolin is Dimebon, a known antihistamine drug that has been used clinically for many years, and has recently shown potential in the treatment of Alzheimer's disease (see, e.g., Doody et al., Lancet 2008; 372: 207-215; Bachurin et al., Annals of the New York Academy of Sciences. 2001; 939: 425-435). Each of the foregoing publications, and each additional publication cited herein, is incorporated herein in their entirety by reference. In addition, described herein are other illustrative dimebolins of formulae (I), (II), (III), (1), and (2). The formulae include various functional groups on aromatic rings, such as R³. It is to be understood that derivatives of those compounds also include the compounds having for example different functional groups on those aromatic rings than those explicitly set forth in the definition of formulae (I), (II), (III), (1), and (2). In addition, it is to be understood that derivatives of those compounds also include the compounds having those same or different functional groups at different positions on the aromatic ring. Similarly, derivatives include parallel variations of other functional groups on the compounds described herein, such as R¹, and the like. In addition, as used herein the term dimebolins also refers to prodrug derivatives of the compounds described herein, and including prodrugs of the various analogs and derivatives thereof.

It is to be understood that certain compounds described herein may also be referred to as analogs and/or derivatives of other compounds described herein. For example, illustrative analogs include, but are not limited to, those compounds that share functional and in some cases structural similarity to those compounds described herein. For example, described herein are illustrative dimebolins of formulae (I), (II), and (III) that include a 2,3,4,5-tetrahydro-1H-pyridoindole ring system. Illustrative analogs include, but are not limited to, the corresponding ring expanded compounds, such as the corresponding azepinoindole ring system, and the like. Other illustrative analogs include, but are not limited to, the corresponding ring systems that include additional heteroatoms, such as the corresponding pyridazinoindole ring system, and the like. Therefore, it is to be understood that all such compounds are also considered to be dimebolins,

Additional illustrative dimebolins that may included in the compositions and methods described herein include hydrogenated pyrido[4,3-b]indoles or pharmaceutically acceptable salts thereof, such as an acid or base salt thereof. A hydrogenated pyrido[4,3-b]indole can be a tetrahydropyrido[4,3-b]indole or pharmaceutically acceptable salt thereof. The hydrogenated pyrido[4,3-b]indole can also be a hexahydropyrido[4,3-b]indole or pharmaceutically acceptable salt thereof. The hydrogenated pyrido[4,3-b]indole compounds can be substituted with 1 to 3 substituents, although unsubstituted hydrogenated pyrido[4,3-b]indole compounds or hydrogenated pyrido[4,3-b]indole compounds with more than 3 substituents are also contemplated. Suitable substituents include but are not limited to alkyl, lower alkyl, arylalkyl, heteroarylalkyl, substituted heteroarylalkyl, substituted arylalkyl, and halo.

The hydrogenated pyrido[4,3-b]indoles can be in the form of pharmaceutically acceptable salts thereof, which are readily known to those of skill in the art. The pharmaceutically acceptable salts include pharmaceutically acceptable acid salts. Examples of particular pharmaceutically acceptable salts include hydrochloride salts or dihydrochloride salts. In a particular variation, the hydrogenated pyrido[4,3-b]indole is a pharmaceutically acceptable salt of 2,8-dimethyl-5-(2-(6-methyl-3-pyridyl)ethyl)-2,3,4,5-tetrahydro-1H-pyrido[4,3-b]indole, such as 2,8-dimethyl-5-(2-(6-methyl-3-pyridyl)ethyl)-2,3,4,5-tetrahydro-1H-pyrido[4,3-b]indole dihydrochloride (dimebon).

In another embodiment, methods are described herein for treating MS that include the step of administering a therapeutically effective amount of one or more dimebolins of formula (I)

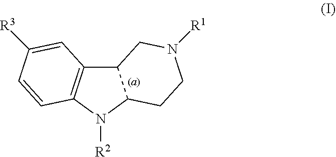

or a pharmaceutically acceptable salt thereof, wherein R¹ is alkyl or arylalkyl; R² is hydrogen, benzyl, or 6-methylpyridinyl-3-ethyl; R³ is hydrogen, alkyl, or halo; and bond (a) is a single bond or a double bond.

In another embodiment, methods are described herein that include the step of administering a therapeutically effective amount of a dimebolin of formula (I) wherein R¹ is methyl, ethyl or benzyl. In another embodiment, methods are described herein that include the step of administering a therapeutically effective amount of a dimebolin of formula (I) wherein R² is hydrogen, benzyl, or 6-methylpyridinyl-3-ethyl. In another embodiment, methods are described herein that include the step of administering a therapeutically effective amount of a dimebolin of formula (I) wherein R³ is hydrogen, methyl, or bromo.

In another embodiment, methods are described herein for treating MS that include the step of administering a therapeutically effective amount of a dimebolin of formula (I) wherein bond (a) is a single bond; R¹ and R³ are each methyl; and R² is hydrogen. In another embodiment, methods are described herein that include the step of administering a therapeutically effective amount of a dimebolin of formula (I) wherein bond (a) is a single bond; and the ring fusion is cis. In another embodiment, methods are described herein that include the step of administering a therapeutically effective amount of a dimebolin of formula (I) wherein bond (a) is a double bond; R¹ is ethyl or benzyl; and R² and R³ are each hydrogen; or R¹ and R³ are each methyl; and R² is benzyl; or R¹ is methyl; R² is 6-methylpyridinyl-3-ethyl; and R³ is hydrogen; or R¹ and R³ are each methyl; and R² is 6-methylpyridinyl-3-ethyl; or R¹ is methyl; R² is hydrogen; and R³ is hydrogen or methyl; or R¹ is methyl; R² is hydrogen; and R³ is bromo.

In another embodiment, methods are described herein that include the step of administering a therapeutically effective amount of a dimebolin of formula (I) where R¹ is selected from the group consisting of alkyl, lower alkyl and arylalkyl, R² is selected from the group consisting of hydrogen, arylalkyl and substituted heteroarylalkyl; and R³ is selected from the group consisting of hydrogen, alkyl, lower alkyl and halo.

In one variation, R¹ is alkyl, such as an alkyl selected from the group consisting of C₁-C₁₅alkyl, C₁₀-C₁₅alkyl, C₁-C₁₀alkyl, C₂-C₁₅alkyl, C₂-C₁₀alkyl, C₂-C₈alkyl, C₄-C₈alkyl, C₆-C₈alkyl, C₆-C₁₅alkyl, C₁₅-C₂₀alkyl; C₁-C₈alkyl and C₁-C₆alkyl. In one variation, R¹ is arylalkyl.

In one variation, R¹ is lower alkyl, such as a lower alkyl selected from the group consisting of C₁-C₂-alkyl, C₁-C₄alkyl, C₂-C₄alkyl, C₁-C₅alkyl, C₁-C₃alkyl, and C₂-C₅alkyl. In one variation, R¹ is a straight chain alkyl group. In one variation, R¹ is a branched alkyl group. In one variation, R¹ is a cyclic alkyl group. In one variation, R¹ is methyl. In one variation, R¹ is ethyl. In one variation, R¹ is methyl or ethyl. In one variation, R¹ is methyl or an arylalkyl group such as benzyl. In one variation, R¹ is ethyl or an arylalkyl group such as benzyl. In one variation, R¹ is an arylalkyl group. In one variation, R¹ is an arylalkyl group where any one of the alkyl or lower alkyl substituents listed in the preceding paragraphs is further substituted with an aryl group (e.g., Ar—C₁-C₆alkyl, Ar—C₁-C₃alkyl or Ar—C₁-C₁₅alkyl). In one variation, R¹ is an arylalkyl group where any one of the alkyl or lower alkyl substituents listed in the preceding paragraphs is substituted with a single ring aryl residue. In one variation, R¹ is an arylalkyl group where any one of the alkyl or lower alkyl substituents listed in the preceding paragraphs is further substituted with a phenyl group (e.g., Ph-C₁-C₆alkyl or Ph-C₁-C₃alkyl, Ph-C₁-C₁₅alkyl). In one variation, R¹ is benzyl. All of the variations for R¹ are intended and hereby clearly described to be combined with any of the variations stated below for R² and R³ the same as if each and every combination of R¹, R² and R³ were specifically and individually listed.

In one variation, R² is H. In one variation, R² is an arylalkyl group. In one variation, R² is a substituted heteroarylalkyl group. In one variation, R² is hydrogen or an arylalkyl group. In one variation, R² is hydrogen or a substituted heteroarylalkyl group. In one variation, R² is an arylalkyl group or a substituted heteroarylalkyl group. In one variation, R² is selected from the group consisting of hydrogen, an arylalkyl group and a substituted heteroarylalkyl group. In one variation, R² is an arylalkyl group where R² can be any one of the arylalkyl groups noted for R¹ above, the same as if each and every arylalkyl variation listed for R¹ is separately and individually listed for R². In one variation, R² is a substituted heteroarylalkyl group, where the alkyl moiety of the heteroarylalkyl can be any alkyl or lower alkyl group, such as those listed above for R¹. In one variation, R² is a substituted heteroarylalkyl where the heteroaryl group is substituted with 1 to 3 C₁-C₃ alkyl substituents (e.g., 6-methyl-3-pyridylethyl). In one variation, R² is a substituted heteroarylalkyl group wherein the heteroaryl group is substituted with 1 to 3 methyl groups. In one variation, R² is a substituted heteroarylalkyl group wherein the heteroaryl group is substituted with one lower alkyl substituent. In one variation, R² is a substituted heteroarylalkyl group wherein the heteroaryl group is substituted with one C₁-C₃ alkyl substituent. In one variation, R² is a substituted heteroarylalkyl group wherein the heteroaryl group is substituted with one or two methyl groups. In one variation, R² is a substituted heteroarylalkyl group wherein the heteroaryl group is substituted with one methyl group.

In other variations, R² is any one of the substituted heteroarylalkyl groups in the immediately preceding paragraph where the heteroaryl moiety of the heteroarylalkyl group is a single ring heteroaryl group. In other variations, R² is any one of the substituted heteroarylalkyl groups in the immediately preceding paragraph where the heteroaryl moiety of the heteroarylalkyl group is a multiple condensed ring heteroaryl group. In other variations, R² is any one of the substituted heteroarylalkyl groups in the immediately preceding paragraph where the heteroarylalkyl moiety is a pyridyl group (Py). In one variation, R² is 6-CH₃-3-Py-(CH₂)₂—. An example of a compound containing this moiety is dimebon.

In one variation, R³ is hydrogen. In other variations, R³ is any one of the alkyl groups noted for R¹ above, the same as if each and every alkyl variation listed for R¹ is separately and individually listed for R. In another variation, R³ is a halo group. In one variation, R³ is hydrogen or an alkyl group. In one variation, R³ is a halo or alkyl group. In one variation, R³ is hydrogen or a halo group. In one variation, R³ is selected from the group consisting of hydrogen, alkyl and halo. In one variation, R³ is Br. In one variation, R³ is I. In one variation, R³ is F. In one variation, R³ is Cl.

In one variation, the compound is of the Formula (I) and R¹ is selected from a lower alkyl or benzyl; R² is selected from a hydrogen, benzyl or 6-CH₃-3-Py-(CH₂)₂— and R is selected from hydrogen, lower alkyl or halo, or any pharmaceutically acceptable salt thereof. In another variation, R¹ is selected from —CH₃, CH₃CH₂—, or benzyl; R² is selected from —H, benzyl, or 6-CH₃-3-Py-(CH₂)₂—; and R³ is selected from —H, —CH₃ or —Br, or any pharmaceutically acceptable salt thereof. In another variation the compound is selected from the group consisting of: cis(±)2,8-dimethyl-2,3,4,4a,5,9b-hexahydro-1H-pyrido[4,3-b]indole as a racemic mixture or in the substantially pure (+) or substantially pure (−) form; 2-ethyl-2,3,4,5-tetrahydro-1H-pyrido[4,3-b]indole; 2-benzyl-2,3,4,5-tetrahydro-1H-pyrido[4,3-b]indole; 2,8-dimethyl-5-benzyl-2,3,4,5-tetrahydro-1H-pyrido[4,3-b]indole; 2-methyl-5-(2-methyl-3-pyridyl)ethyl-2,3,4,5-tetrahydro-1H-pyrido[4,3-b]indole; 2,8-dimethyl-5-(2-(6-methyl-3-pyridyl)ethyl)-2,3,4,5-tetrahydro-1H-pyrido[4,3-b]indole; 2-methyl-2,3,4,5-tetrahydro-1H-pyrido[4,3-b]indole; 2,8-dimethyl-2,3,4,5-tetrahydro-1H-pyrido[4,3-b]indole; or 2-methyl-8-bromo-2,3,4,5-tetrahydro-1H-pyrido[4,3-b]indole or any pharmaceutically acceptable salt of any of the foregoing. In one variation, the compound is of the formula A or B wherein R¹ is —CH₃, R² is —H and R³ is —CH₃ or any pharmaceutically acceptable salt thereof. The compound may be of the Formula A or B where R¹ CH₃CH₂— or benzyl, R² is —H, and R³ is —CH₃ or any pharmaceutically acceptable salt thereof. The compound may be of the Formula A or B where R¹ is —CH₃, R² is benzyl, and R³ is —CH₃ or any pharmaceutically acceptable salt thereof. The compound may be of the Formula A or B where R¹ is —CH₃, R² is 6-CH₃-3-Py-(CH₂)₂—, and R³ is —H or any pharmaceutically acceptable salt thereof. The compound may be of the Formula A or B where R² is 6-CH₃-3-Py-(CH₂)₂— or any pharmaceutically acceptable salt thereof. The compound may be of the Formula A or B where R¹ is —CH₃, R² is —H, and R³ is —H or —CH₃ or any pharmaceutically acceptable salt, thereof. The compound may be of the Formula A or B where R¹ is —CH₃, R² is —H, and R³ is —Br, or any pharmaceutically acceptable salt thereof. The compound may be of the Formula A or B where R¹ is selected from a lower alkyl or arylalkyl, R² is selected from a hydrogen, arylalkyl or substituted heteroarylalkyl and R³ is selected from hydrogen, lower alkyl or halo.

In another embodiment, methods are described herein for treating MS that include the step of administering a therapeutically effective amount of one or more dimebolins of formula (II)

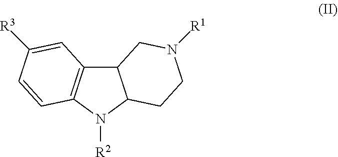

or a pharmaceutically acceptable salt thereof, wherein R¹ is alkyl or arylalkyl; R² is hydrogen, benzyl, or 6-methylpyridinyl-3-ethyl; R³ is hydrogen, alkyl, or halo; and bond (a) is a single bond or a double bond.

In another embodiment, methods are described herein for treating MS that include the step of administering a therapeutically effective amount of a dimebolin of formula (II) wherein R¹ is methyl, ethyl or benzyl. In another embodiment, methods are described herein that include the step of administering a therapeutically effective amount of a dimebolin of formula (II) wherein R² is hydrogen, benzyl, or 6-methylpyridinyl-3-ethyl. In another embodiment, methods are described herein that include the step of administering a therapeutically effective amount of a dimebolin of formula (II) wherein R³ is hydrogen, methyl, or bromo. In another embodiment, methods are described herein include the step of administering a therapeutically effective amount of a dimebolin of formula (II) wherein R¹ and R³ are each methyl; and R² is hydrogen. In another embodiment, methods are described herein that include the step of administering a therapeutically effective amount of a dimebolin of formula (II) wherein the ring fusion is cis. In another embodiment, methods are described herein that include the step of administering a therapeutically effective amount of a dimebolin of formula (II) in a pharmaceutically acceptable quaternary salt form.

In another embodiment, methods are described herein for treating MS that include the step of administering a therapeutically effective amount of one or more dimebolins of formula (III)

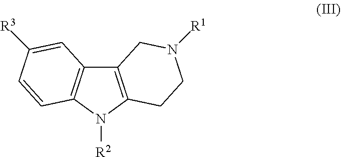

or a pharmaceutically acceptable salt thereof, wherein R¹ is alkyl or arylalkyl; R² is hydrogen, benzyl, or 6-methylpyridinyl-3-ethyl; R³ is hydrogen, alkyl, or halo; and bond (a) is a single bond or a double bond.

In another embodiment, methods are described herein for treating MS that include the step of administering a therapeutically effective amount of one or more dimebolins of formula (III) wherein R¹ is methyl, ethyl or benzyl. In another embodiment, methods are described herein that include the step of administering a therapeutically effective amount of one or more dimebolins of formula (III) wherein R² is hydrogen, benzyl, or 6-methylpyridinyl-3-ethyl. In another embodiment, methods are described herein that include the step of administering a therapeutically effective amount of one or more dimebolins of formula (III) wherein R³ is hydrogen, methyl, or bromo.

In another embodiment, methods are described herein for treating MS that include the step of administering a therapeutically effective amount of one or more dimebolins of formula (III) wherein R¹ is ethyl or benzyl; and R² and R³ are each hydrogen; or R¹ and R³ are each methyl; and R² is benzyl; or R¹ is methyl; R² is 6-methylpyridinyl-3-ethyl; and R³ is hydrogen; or R¹ and R³ are each methyl; and R² is 6-methylpyridinyl-3-ethyl; or R¹ is methyl; R² is hydrogen; and R³ is hydrogen or methyl; or R¹ is methyl; R² is hydrogen; and R³ is bromo. In another embodiment, methods are described herein that include the step of administering a therapeutically effective amount of one or more dimebolins of formula (III) in a pharmaceutically acceptable quaternary salt form.

Additional illustrative dimebolins that may included in the compositions and methods described herein include hydrogenated pyrido[4,3-b]indoles of the Formula (1) or the Formula (2):

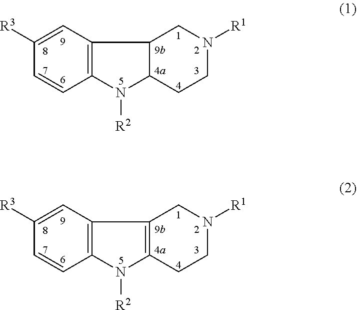

For compounds of a general Formula (1) or (2), R¹ represents —CH₃, CH₃CH₂—, or PhCH₂-(benzyl); R² is —H, PhCH₂—, or 6CH₃-3-Py-(CH₂)₂—; R³ is —H, —CH₃, or —Br, in any combination of the above substituents. All possible combinations of the substituents of Formula (1) and (2) are contemplated as specific and individual compounds the same as if each single and individual compound were listed by chemical name. Also contemplated are the compounds of Formula (1) or (2), with any deletion of one or more possible moieties from the substituent groups listed above: e.g., where R¹ represents —CH₃. In one variation, R² is —H, PhCH₂—, or 6CH₃-3-Py-(CH₂)₂—; and R³ is —H, —CH₃, or —Br, or where R¹ represents —CH₃; R² is 6CH₃-3-Py-(CH₂)₂—; and R³ represents —H, —CH₃, or —Br.

The compound may be Formula (1), where R¹ is —CH₃, R² is —H, and R³ is —CH₃. The compound may be Formula (2), where R¹ is represented by —CH₃, CH₃CH₂—, or PhCH₂—; R² is —H, PhCH₂—, or 6CH₃-3-Py-(CH₂)₂—; R³ is —H, —CH₃, or —Br. The compound may be Formula (2), where R¹ is CH₃CH₂— or PhCH₂—, R² is —H, and R³ is —H; or a compound, where R¹ is —CH₃, R² is PhCH₂—, R³ is —CH₃; or a compound, where R¹ is —CH₃, R² is 6-CH₃-3-Py-(CH₂)₂—, and R³ is —CH₃; or a compound, where R¹ is —CH₃, R² is —H, R³ is —H or —CH₃; or a compound, where R¹ is —CH₃, R² is —H, R³ is —Br.

In another embodiment, methods are described herein for treating MS that include the step of administering a therapeutically effective amount of a dimebolin of the formula

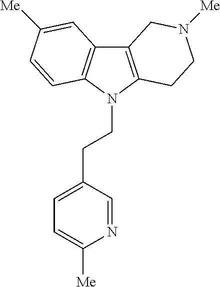 [SMILES: Cc1ccc2c(c1)c1c(n2CCc2ccc(C)nc2)CCN(C)C1]

or a pharmaceutically acceptable salt, such as the hydrochloride salt.

In another embodiment, methods are described herein for treating MS that include the step of administering a therapeutically effective amount of one or more compounds selected from 2-methyl-2,3,4,5-tetrahydro-1H-pyrido[4,3-b]indole; 2,8-dimethyl-2,3,4,5-tetrahydro-1H-pyrido[4,3-b]indole, or its methyliodide; cis-(±) 2,8-dimethyl-2,3,4,4a,5,9b-hexahydro1H-pyrido[4,3-b]indole, or its dihydrochloride; 2-methyl-8-bromo-2,3,4,5-tetrahydro-1H-pyrido[4,3-b]indole, or its hydrochloride; 2-ethyl-2,3,4,5-tetrahydro-1H-pyrido[4,3-b]indole; 2-benzyl-2,3,4,5-tetrahydro-1H-pyrido[4,3-b]indole; 2,8-dimethyl-5-benzyl-2,3,4,5-tetrahydro-1H-pyrido[4,3-b]indole, or its hydrochloride; 2-methyl-5-[2-(6-methyl-3-pyridyl)ethyl]-2,3,4,5-tetrahydro-1H-pyrido[4,3-b]indole, or its sesquisulfate monohydrate; and 2,8-dimethyl-5-[2-(6-methyl-3-pyridyl)ethyl]-2,3,4,5-tetrahydro-1H-pyrido[4,3-b]indole, or its dihydrochloride. The foregoing compounds may be prepared according to Horlein, Chem. Ber., 1954, Bd.87, hft 4, p. 463-472; Cattanach et al., J. Chem. Soc. (ser. C) 1968, 1235-1243; Yurovskaya and Rodionov, Khim. Geterots. Soed., 1981, No. 8, p. 1072-1078; Yakhontov and Glushkova, Synthatic Drugs (edited by A. G. Natradze), Moscow, “Meditsina Publishers”, 1983, p. 234-237; Buu-Hoi et al., J. Chem. Soc., 1964, No. 2, p. 708-711; Kucherova and Kochetkov, J. Obshch. Khim., 1956, v. 26, p. 3149-3154; and Kost et al., “Khim. Geterots. Soed.”, 1973, No. 2, p. 207-212, the disclosure of which are incorporated herein by reference.

Additional illustrative dimebolins that may included in the compositions and methods described herein include: cis(±) 2,8-dimethyl-2,3,4,4a,5,9b-hexahydro-1H-pyrido[4,3-b]indole and its dihydrochloride; 2-ethyl-2,3,4,5-tetrahydro-1H-pyrido[4,3-b]indole; 2-benzyl-2,3,4,5-tetrahydro-1H-pyrido[4,3-b]indole; 2,8-dimethyl-5-benzyl-2,3,4,5-tetrahydro-1H-pyrido[4,3-b]indole and its dihydrochloride; 2-methyl-5-(2-methyl-3-pyridyl)ethyl-2,3,4,5-tetrahydro-1H-pyrido[4,3-b]indole and its sesquisulfate; 2,8-dimethyl-5-(2-(6-methyl-3-pyridyl)ethyl)-2,3,4,5-tetrahydro-1H-pyrido[4,3-b]indole and its dihydrochloride (dimebon); 2-methyl-2,3,4,5-tetrahydro-1H-pyrido[4,3-b]indole; 2,8-dimethyl-2,3,4,5-tetrahydro-1H-pyrido[4,3-b]indole and its methyl iodide; 2-methyl-8-bromo-2,3,4,5-tetrahydro-1H-pyrido[4,3-b]indole and its hydrochloride, as described in the following Compounds listed above as compounds 1-9 from the literature are detailed in the following publications, each incorporated herein by reference in it entirety. Synthesis and studies on neuroleptic properties for cis(±)2,8-dimethyl-2,3,4,4a,5,9b-hexahydro-1H-pyrido[4,3-b]indole and its dihydrochloride are reported, for instance, in the following publication: Yakhontov, L. N., Glushkov, R. G., Synthetic Therapeutic Drugs; A. G. Natradze, Ed., Moscow Medicina, 1983, p. 234-237. Synthesis of compounds 2, 8, and 9 above, and data on their properties as serotonin antagonists are reported in, for instance, in C. J. Cattanach, A. Cohen & B. H. Brown, J. Chem. Soc. (Ser. C) 1968, p. 1235-1243. Synthesis of the compound 3 above is reported, for instance, in the article N. P. Buu-Hoi, O. Roussel, P. Jacquignon, J. Chem. Soc, 1964, N 2, p. 708-711. N. F. Kucherova and N. K. Kochetkov (General Chemistry (Russ.), 1956, 26:3149-3154) describe the synthesis of the compound 4 above. Synthesis of compounds 5 and 6 above is described in the article by A. N. Kost, M. A. Yurovskaya, T. V. Mel'nikova, in Chemistry of Heterocyclic Compounds, 1973, N 2, p. 207-212. The synthesis of the compound 7 above is described by U, Horlein in Chem. Ber., 1954, Bd. 87, hft 4, 463-p. 472. M. Yurovskaya and LL. Rodionov in Chemistry of Heterocyclic Compounds (1981, N 8, p. 1072-10).

In a another variation, the hydrogenated pyrido[4,3-b]indole is 2,8-dimethyl-5-(2-(6-methyl-3-pyridyl)ethyl)-2,3,4,5-tetrahydro-1H-pyrido[4,3-b]indole or a pharmaceutically acceptable salt thereof. The compound for use in the compositions and methods may be 2,8-dimethyl-5-(2-(6-methyl-3-pyridyl)ethyl-2,3,4,5-tetrahydro-1H-pyrido[4,3-b]indole or any pharmaceutically acceptable salt thereof, such as an acid salt, a hydrochloride salt or a dihydrochloride salt thereof.

It is to be understood that any of the compounds disclosed herein having two stereocenters in the pyrido[4,3-b]indole ring structure (e.g., carbons 4a and 9b of compound (I)) includes compounds whose stereocenters are in a cis or a trans form. A composition may comprise such a compound in substantially pure form, such as a composition of substantially pure S, S or R,R or S,R or R,S compound. A composition of substantially pure compound means that the composition contains no more than 15% or no more than 10% or no more than 5% or no more than 3% or no more than 1% impurity of the compound in a different stereochemical form. For instance, a composition of substantially pure S,S compound means that the composition contains no more than 15% or no more than 10% or no more than 5% or no more than 3% or no more than 1% of the R,R or S,R or R,S form of the compound. A composition may contain the compound as mixtures of such stereoisomers, where the mixture may be enantiomers (e.g., S,S and R,R) or diastereomers (e.g., S,S and R,S or S,R) in equal or unequal amounts. A composition may contain the compound as a mixture of 2 or 3 or 4 such stereoisomers in any ratio of stereoisomers. Compounds disclosed herein having stereocenters other than in the pyrido[4,3-b]indole ring structure intends all stereochemical variations of such compounds, including but not limited to enantiomers and diastereomers in any ratio, and includes racemic and enantioenriched and other possible mixtures. Unless stereochemistry is explicitly indicated in a structure, the structure is intended to embrace all possible stereoisomers of the compound depicted.

It is to be understood that in any of the method or composition embodiments described herein, the corresponding acid addition salt may be administered in addition to or instead of the corresponding neutral compound. Illustrative acid salts may be formed from, but at not limited to, inorganic acids such as hydrohalic acids, including hydrochloric or hydrobromic acid; sulfuric; nitric; phosphoric, and the like acids; and organic acids such as acetic, propanoic, hydroxyacetic, lactic, pyruvic, oxalic, malonic, succinic, maleic, fumaric, malic, tartaric, citric, methanesulfonic, ethanesulfonic, benzenesulfonic, p-toluenesulfonic, cyclamic, salicylic, p-aminosalicylic, pamoic and the like acids. In addition, it is to be understood that bis salts may be formed and administered, such as the dihydrochloride acid salt, and the like.

As used herein, the term “aryl” includes monocyclic and polycyclic aromatic carbocyclic and aromatic heterocyclic groups, each of which may be optionally substituted. As used herein, the term “heteroaryl” includes aromatic heterocyclic groups, each of which may be optionally substituted. Illustrative carbocyclic aromatic groups described herein include, but are not limited to, phenyl, naphthyl, and the like. Illustrative heterocyclic aromatic groups include, but are not limited to, pyridinyl, pyrimidinyl, pyrazinyl, triazinyl, tetrazinyl, quinolinyl, quinazolinyl, quinoxalinyl, thienyl, pyrazolyl, imidazolyl, oxazolyl, thiazolyl, isoxazolyl, isothiazolyl, oxadiazolyl, thiadiazolyl, triazolyl, benzimidazolyl, benzoxazolyl, benzthiazolyl, benzisoxazolyl, benzisothiazolyl, and the like.

As used herein, the term “amino” includes the group NH₂, alkylamino, and dialkylamino, where the two alkyl groups in dialkylamino may be the same or different, i.e. alkylalkylamino. Illustratively, amino include methylamino, ethylamino, dimethylamino, methylethylamino, and the like. In addition, it is to be understood that when amino modifies or is modified by another term, such as aminoalkyl, or acylamino, the above variations of the term amino continue to apply. Illustratively, aminoalkyl includes H₂N-alkyl, methylaminoalkyl, ethylaminoalkyl, dimethylaminoalkyl, methylethylaminoalkyl, and the like. Illustratively, acylamino includes acylmethylamino, acylethylamino, and the like.

The term “prodrug” as used herein generally refers to any compound that when administered to a biological system generates a biologically active compound as a result of one or more spontaneous chemical reaction(s), enzyme-catalyzed chemical reaction(s), and/or metabolic chemical reaction(s), or a combination thereof. In vivo, the prodrug is typically acted upon by an enzyme (such as esterases, amidases, phosphatases, and the like), simple biological chemistry, or other process in vivo to liberate or regenerate the more pharmacologically active drug. This activation may occur through the action of an endogenous host enzyme or a non-endogenous enzyme that is administered to the host preceding, following, or during administration of the prodrug. Additional details of prodrug use are described in U.S. Pat. No. 5,627,165; and Pathalk et al., Enzymic protecting group techniques in organic synthesis, Stereosel. Biocatal. 775-797 (2000). It is appreciated that the prodrug is advantageously converted to the original drug as soon as the goal, such as targeted delivery, safety, stability, and the like is achieved, followed by the subsequent rapid elimination of the released remains of the group forming the prodrug.

Prodrugs may be prepared from the compounds described herein by attaching groups that ultimately cleave in vivo to one or more functional groups present on the compound, such as OH, SH, CO₂H, NR₂. Illustrative prodrugs include but are not limited to carboxylate esters where the group is alkyl, aryl, aralkyl, acyloxyalkyl, alkoxycarbonyloxyalkyl as well as esters of hydroxyl, thiol and amines where the group attached is an acyl group, an alkoxycarbonyl, aminocarbonyl, phosphate or sulfate. Further illustrative prodrugs contain a chemical moiety, such as an amide or phosphorus group functioning to increase solubility and/or stability of the compounds described herein. Further illustrative prodrugs for amino groups include, but are not limited to, (C₃-C₂₀)alkanoyl; halo-(C₃-C₂₀)alkanoyl; (C₃-C₂₀)alkenoyl; (C₄-C₇)cycloalkanoyl; (C₃-C₆)-cycloalkyl(C₂-C₁₆)alkanoyl; optionally substituted aroyl, such as unsubstituted aroyl or aroyl substituted by 1 to 3 substituents selected from the group consisting of halogen, cyano, trifluoromethanesulphonyloxy, (C₁-C₃)alkyl and (C₁-C₃)alkoxy, each of which is optionally further substituted with one or more of 1 to 3 halogen atoms; optionally substituted aryl(C₂-C₁₆)alkanoyl, such as the aryl radical being unsubstituted or substituted by 1 to 3 substituents selected from the group consisting of halogen, (C₁-C₃)alkyl and (C₁-C₃)alkoxy, each of which is optionally further substituted with 1 to 3 halogen atoms; and optionally substituted heteroarylalkanoyl having one to three heteroatoms selected from 0, S and N in the heteroaryl moiety and 2 to 10 carbon atoms in the alkanoyl moiety, such as the heteroaryl radical being unsubstituted or substituted by 1 to 3 substituents selected from the group consisting of halogen, cyano, trifluoromethanesulphonyloxy, (C₁-C₃)alkyl, and (C₁-C₃)alkoxy, each of which is optionally further substituted with 1 to 3 halogen atoms. The groups illustrated are exemplary, not exhaustive, and may be prepared by conventional processes.

It is understood that the prodrugs themselves may not possess significant biological activity, but instead undergo one or more spontaneous chemical reaction(s), enzyme-catalyzed chemical reaction(s), and/or metabolic chemical reaction(s), or a combination thereof after administration in vivo to produce the compound described herein that is biologically active or is a precursor of the biologically active compound. However, it is appreciated that in some cases, the prodrug is biologically active. It is also appreciated that prodrugs may often serves to improve drug efficacy or safety through improved oral bioavailability, pharmacodynamic half-life, and the like. Prodrugs also refer to derivatives of the compounds described herein that include groups that simply mask undesirable drug properties or improve drug delivery. For example, one or more compounds described herein may exhibit an undesirable property that is advantageously blocked or minimized may become pharmacological, pharmaceutical, or pharmacokinetic barriers in clinical drug application, such as low oral drug absorption, lack of site specificity, chemical instability, toxicity, and poor patient acceptance (bad taste, odor, pain at injection site, and the like), and others. It is appreciated herein that a prodrug, or other strategy using reversible derivatives, can be useful in the optimization of the clinical application of a drug.

As used herein, the term “composition” generally refers to any product comprising the specified ingredients in the specified amounts, as well as any product which results, directly or indirectly, from combinations of the specified ingredients in the specified amounts. Illustratively, compositions may include one or more carriers, diluents, and/or excipients. The compounds described herein may be formulated in a therapeutically effective amount in conventional dosage forms for the methods described herein, including one or more carriers, diluents, and/or excipients therefor. Such formulation compositions may be administered by a wide variety of conventional routes for the methods described herein in a wide variety of dosage formats, utilizing art-recognized products. See generally, Remington's Pharmaceutical Sciences, (16th ed. 1980). It is to be understood that the compositions described herein may be prepared from isolated compounds described herein or from salts, solutions, hydrates, solvates, and other forms of the compounds described herein. It is also to be understood that the compositions may be prepared from various amorphous, non-amorphous, partially crystalline, crystalline, and/or other morphological forms of the compounds described herein.

The term “therapeutically effective amount” as used herein, refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the disorder being treated and the severity of the disorder; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known in the medical arts.

The term “administering” as used herein includes both systemic use and local use of the compositions described herein. Systemic use includes compositions and methods that include oral routes, parenteral routes (including subcutaneous, intramuscular, intravenous and intrathecal routes), inhalation spray routes, nasal, ocular, rectal, sublingual, and buccal routes, and topical routes. In each route, the compositions are in dosage form unit formulations, and may contain one or more conventional nontoxic pharmaceutically-acceptable carriers, adjuvants, excipients, diluents, and vehicles. Local use includes compositions and methods that include locally parenteral routes directly or indirectly delivered to the site of disease, injury, or defect. Illustrative local administration may be performed during open surgery, or other procedures when the site of disease, injury, or defect is accessible. Alternatively, local administration may be performed using parenteral delivery where the compound or compositions described herein are deposited locally to the site without general distribution to multiple other non-target sites in the patient being treated. It is further appreciated that local administration may be directly in the injury site, or locally in the surrounding tissue. Similar variations regarding local delivery to particular tissue types, such as organs, and the like, are also described herein.

In another embodiment, compositions and methods are described herein where the therapeutically effective amount is capable of blocking mitochondrial permeability transition pores (MPTPs). Without being bound by theory, it is believed herein that in contrast to current hypotheses, the therapeutic potential in the treatment of MS using dimebolins, and pharmaceutically acceptable salts thereof, is based at least in part, or may be related to the ability of those compounds to inhibit MPTPs. It is appreciated that inhibiting and/or blocking MPTPs, such as preventing pores from opening, or decreasing the number of pores that open, may lead to mitochondrial stabilization. In another embodiment, methods are described herein where the therapeutically effective amount is capable antagonizing NMDA receptors. Without being bound by theory, it is believed herein that the therapeutic potential in the treatment of MS using dimebolins, and pharmaceutically acceptable salts thereof, is based at least in part, or may be related to the ability of dimebolins, and pharmaceutically acceptable salts thereof, to antagonize NMDA receptors or selectively antagonize NMDA receptors. In addition, though without being bound by theory, it is believed herein that the therapeutic potential in the treatment of MS using dimebolins, and pharmaceutically acceptable salts thereof, is based at least in part, or may be related to the ability of dimebolins, and pharmaceutically acceptable salts thereof, to inhibit MPTPs, to antagonize NMDA receptors or selectively antagonize NMDA receptors, and/or a combination of the foregoing, which has the consequence of decreasing or preventing axonal damage, of decreasing or preventing neuronal death, such as excitotoxic neuronal death, of decreasing or preventing over-stimulation of NMDA receptors leading to excessive mitochondrial calcium accumulation and damage, and/or a combination of the foregoing. In addition, though without being bound by theory, it is believed herein that the therapeutic potential in the treatment of MS using dimebolins, and pharmaceutically acceptable salts thereof, is based at least in part, or may be related to the ability of dimebolins, and pharmaceutically acceptable salts thereof, as neuroprotectants, such as oligodendrocyte protectants. Accordingly, it is appreciated that such neuroprotection may protect axons and allow endogenous processes to more effectively repair myelin.

Nevertheless, it is appreciated that blockade of all NMDA sites may be accompanied by several unwanted side effects, such as has been observed with PCP narcotics, and other non-selective NMDA antagonists. It is therefore understood that activity at selective and/or specific subunits may be advantageous in the treatment of MS, and it is appreciated that dimebolins and pharmaceutically acceptable salts thereof may have such selective and/or specific activity.

As used herein the term “multiple sclerosis” or MS includes neurological disorders characterized by demyelination, including neurological disorders characterized by an autoimmune reaction leading to demyelination. It is understood that MS may cause numerous physical and mental symptoms, and often progresses to physical and cognitive disability. Disease onset usually occurs in young adults, and MS has been reported to be more common in women. MS has been reported to have a prevalence that ranges between 2 and 150 per 100,000 depending on the country or specific population (see, e.g., Rosati G (April 2001) Neurol. Sci. 22 (2): 117-39. PMID 1160361426).

MS affects the areas of the brain and spinal cord known as the white matter. White matter cells carry signals between the grey matter areas, where the processing is done, and the rest of the body. MS results in a thinning or complete loss of myelin and in an axonal damage. When the myelin is lost, the neurons can no longer effectively conduct neural impulses. More specifically, the destruction of oligodendrocytes, glial cells that are the cells responsible for creating and maintaining the neuronal myelin sheath, is generally accompanied by MS. Without being bound by theory, it is believed herein that either MS causes the destruction of oligodendrocytes by causing mitochondrial permeability transition pores to open, or that as a consequence of demyelination by another mechanism, mitochondrial permeability transition pores open, leading to the ultimate death of the oligodendrocytes.

MS takes several forms, with new symptoms occurring either in discrete attacks (relapsing forms) or slowly accumulating over time (progressive forms). Most people are first diagnosed with relapsing-remitting MS but develop secondary-progressive MS (SPMS) after a number of years. Between attacks, symptoms may go away completely, but permanent neurological problems often persist, especially as the disease advances.

Although much is known about the mechanisms involved in the disease process, the underlying cause or triggering of MS remains unknown. A well-reported theory is that the condition is autoimmune related. However, it has also been reported on the one hand that the disease is a metabolically dependent disease while on the other hand that it is caused by a virus such as Epstein-Barr. Still other theories have been reported, including that based on its virtual absence from tropical areas, MS may arise from a deficiency of vitamin D during childhood. Regardless of its cause, there is not current cure for MS, but several therapies have proven helpful. In each case, the treatments attempt to return function after an attack, prevent new attacks, and/or prevent worsening or progression of the disease leading to disability. The prognosis for successful therapy depends on the subtype of the disease, the individual patient's disease characteristics, the initial symptoms, and the degree of disability the person experiences as time advances.

In contrast, though without being bound by theory, it is believed herein that one illustrative characteristic of dimebolins, including analogs of the compounds described herein, is MPTP inhibition. It is further believed herein that mitochondrial dysfunction may play an important role in the development of axonal damage and/or neuronal death, which occurs in all types of multiple sclerosis but may be especially prevalent in the primary and secondary-progressive forms. Without being bound by theory, it is believed herein that another illustrative characteristic of the dimebolins, including analogs of the compounds described herein, is NMDA antagonism. Without being bound by theory, it is also believed herein that another illustrative characteristic of the dimebolins, including analogs of the compounds described herein, is the observed pharmacokinetic characteristics and ability to cross the blood-brain-barrier, in conjunction with a low adverse event profile (Doody et al., Lancet. 2008 Jul. 19; 372(9634):207-15).

However, it is also appreciated herein that not all MPTP blockers and or NMDA antagonists will have the same potential in treating MS, especially non-selctive NMDA antagonists. For example, a well known antagonist of mitochondrial permeability transition pores is cyclosporin A (see, e.g., Sullivan et al., Experimental Neurology. Volume 161, Issue 2, Pages 631-637), but long term therapy using cyclosporin A is limited due to the strong induction of immunosuppression and accompanying toxicity (see, e.g., Magnasco et al., Curr Clin Pharmacol. 2008 September; 3(3):166-73. Another well known NMDA receptor antagonist is 2-amino-5-phosphonovaleric acid (AP5) (Evans et al., —Brit. J. Pharmacol., 1982, v.75, p.65). However, AP5 has been reported to suffer from the disadvantage of having neurotoxic effects, including disturbance of coordination of movement and a sedative effect, each of which becomes apparent when AP5 is used in the doses in which it produces its anti-NMDA effect (ED50=190 mg/kg) (Grigoriev et al. Chim. Pharm. Journal, 1988, No. 3, p. 275-277). In addition, unacceptable adverse event profiles have been reported for other MPTP blockers, such as cyclosporin A and bongkrekic (bongkrek) acid (see, e.g., Peter et al., Journal of Biological Chemistry, (1970) 245, 6, 1319). Similarly, most other NMDA antagonists may either have an unacceptable adverse prevent profile, such as has been observed with aptiganel, phencyclidine, and remacemide, unfavorable pharmacokinetic characteristics, such as exhibited by MRZ 2/596 and MDL 105,519, or reduced efficacy, such as has been observed with remacemide.

In another embodiment, compositions and methods are described herein that also include one or more additional NMDA antagonists. Illustratively, the additional NMDA antagonist is an uncompetitive channel blocker. Illustrative uncompetitive channel blockers include, but are not limited to, riluzole, memantine, amantadine, dextromethorphan, dextrorphan, ibogaine, ketamine, phencyclidine, tiletamine, remacemide, and the like, and pharmaceutically acceptable salts thereof. In one variation, the additional NMDA receptor antagonists are selected from riluzole, memantine, amantadine, and dextromethorphan, and pharmaceutically acceptable salts thereof. In another variation, the additional NMDA receptor antagonist is memantine (also known as AXURA, AKATINOL, NAMENDA, EBIXA, and 1-amino-3,5-dimethylada-mantane), or a pharmaceutically acceptable salts thereof. Illustratively, the additional NMDA antagonist is a noncompetitive antagonist. Illustrative noncompetitive antagonists include, but are not limited to, HU-211, Dizocilpine (MK-801), aptiganel (CERESTAT, CNS-1102), remacemide, and the like, and pharmaceutically acceptable salts thereof. Illustratively, the additional NMDA antagonist is a glycine antagonist, such as a compound that binds to and/or operates through an allosteric site of action at the glycine binding site. Illustrative glycine antagonists include, but are not limited to, 7-chlorokynurenate, 5,7-dichlorokynurenic acid (DCKA), kynurenic acid, 1-aminocyclopropanecarboxylic acid (ACPC), and the like, and pharmaceutically acceptable salts thereof.

In another embodiment, compositions and methods are described herein that also include one or more HMG-CoA reductase inhibitors. Illustrative HMG-CoA reductase inhibitors include, but are not limited to, simvastatin, lovastatin, pravastatin, fluvastatin, atorvastatin, rosuvastatin, cerivastatin, and the like, and pharmaceutically acceptable salts thereof. In one variation, the HMG-CoA reductase inhibitor is simvastatin and/or lovastatin, or a pharmaceutically acceptable salt thereof.

In another embodiment, compositions and methods are described herein that also include one or more immunosuppressive drugs. Illustrative immunosuppressive drugs include, but are not limited to, azathioprine, mycophenolate mofetil, corticosteroids, mitoxantrone, cyclophophosphamide, methotrexate, cyclosporine, and the like, and pharmaceutically acceptable salts thereof. It is appreciated that some immunosuppressive drugs are monoclonal antibodies. In one variation, illustrative immunosuppressive drugs are monoclonal antibodies. Illustrative immunosuppressive drugs include, but are not limited to, natalizumab, daclizumab, alemtuzumab, rituximab, and the like. In another variation, the immunosuppressive drug is azathioprine, or pharmaceutically acceptable salts thereof.

In another embodiment, compositions and methods are described herein that also include one or more immunomodulatory drugs. Illustrative immunomodulatory drugs include, but are not limited to, interferon beta-1b, interferon beta-1a, glatiramer acetate (copaxone), natalizumab, rituximab, daclizumab, BG12, fingolimod, laquinimod, and the like, and pharmaceutically acceptable salts thereof. It is appreciated that some immunomodulatory drugs are monoclonal antibodies. In one variation, illustrative immunomodulatory drugs are monoclonal antibodies. Illustrative immunomodulatory drugs include, but are not limited to, natalizumab, daclizumab, alemtuzumab, rituximab, and the like.

In another embodiment, one or more dimebolins and/or pharmaceutically acceptable salts thereof is co-administered with simvastatin and glatiramer acetate.

It is to be understood that in the methods described herein, the individual components of a coadministration, or combination can be administered by any suitable means, simultaneously, sequentially, separately or in a single pharmaceutical formulation. Illustratively, where one or more dimebolins and/or pharmaceutically acceptable salts thereof, one or more NMDA receptor antagonists, one or more statins, one or more immunomodulatory drugs, and/or one or more immunosuppressive drugs are administered in separate dosage forms, the number of dosages administered per day for each compound may be the same or different. One or more dimebolins and/or pharmaceutically acceptable salts thereof, and optionally one or more NMDA receptor antagonists and/or one or more statins and/or one or more immunomodulatory drugs and/or one or more immunosuppressive drugs may be administered via the same or different routes of administration. The compounds or compositions may be administered according to simultaneous or alternating regimens, at the same or different times during the course of the therapy, concurrently in divided or single forms.

In another illustrative embodiment where either or both of an NMDA receptor antagonist and/or an immunosuppressive drug are co-administered with one or more dimebolins and/or pharmaceutically acceptable salts thereof, co-administration includes dosing protocols where the two or more compounds are given simultaneously or contemporaneously. It is to be understood that co-administration is not limited to any particular time frame. For example, dosing protocols where one or more dimebolins and/or pharmaceutically acceptable salts thereof are given every other day, and the NMDA antagonist is given on the alternate days that one or more dimebolins and/or pharmaceutically acceptable salts thereof are not given are included in the co-administration methods described herein. In another illustrative embodiment where either or both of an NMDA receptor antagonist and/or an immunomodulatory drug are co-administered with one or more dimebolins and/or pharmaceutically acceptable salts thereof, co-administration includes dosing protocols where the two or more compounds are given simultaneously or contemporaneously.

In making the pharmaceutical compositions of the compounds described herein, a therapeutically effective amount of one or more compounds in any of the various forms described herein may be mixed with one or more excipients, diluted by one or more excipients, or enclosed within such a carrier which can be in the form of a capsule, sachet, paper, or other container. Excipients may serve as a diluent, and can be solid, semi-solid, or liquid materials, which act as a vehicle, carrier or medium for the active ingredient. Thus, the formulation compositions can be in the form of tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, and sterile packaged powders. The compositions may contain anywhere from about 0.1% to about 99.9% active ingredients, depending upon the selected dose and dosage form. Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include lubricating agents such as talc, magnesium stearate, and mineral oil; wetting agents; emulsifying and suspending agents; preserving agents such as methyl- and propylhydroxybenzoates; sweetening agents; and flavoring agents. The compositions can be formulated so as to provide quick, sustained or delayed release of the active ingredient after administration to the patient by employing procedures known in the art. It is appreciated that the carriers, diluents, and excipients used to prepare the compositions described herein are advantageously GRAS (Generally Regarded as Safe) compounds.

Examples of emulsifying agents are naturally occurring gums (e.g., gum acacia or gum tragacanth) and naturally occurring phosphatides (e.g., soybean lecithin and sorbitan monooleate derivatives). Examples of antioxidants are butylated hydroxy anisole (BHA), ascorbic acid and derivatives thereof, tocopherol and derivatives thereof, butylated hydroxy anisole, and cysteine. Examples of preservatives are parabens, such as methyl or propyl p-hydroxybenzoate, and benzalkonium chloride. Examples of humectants are glycerin, propylene glycol, sorbitol, and urea. Examples of penetration enhancers are propylene glycol, DMSO, triethanolamine, N,N-dimethylacetamide, N,N-dimethylformamide, 2-pyrrolidone and derivatives thereof, tetrahydrofurfuryl alcohol, and AZONE. Examples of chelating agents are sodium EDTA, citric acid, and phosphoric acid. Examples of gel forming agents are CARBOPOL, cellulose derivatives, bentonite, alginates, gelatin and polyvinylpyrrolidone. Examples of ointment bases are beeswax, paraffin, cetyl palmitate, vegetable oils, sorbitan esters of fatty acids (Span), polyethylene glycols, and condensation products between sorbitan esters of fatty acids and ethylene oxide (e.g., polyoxyethylene sorbitan monooleate (TWEEN)).

Solid Dosage Forms for Oral Use. Formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active drug substance in a predetermined pattern (e.g., in order to achieve a controlled release formulation) or it may be adapted not to release the active drug substance until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material such as, e.g., glyceryl monostearate or glyceryl distearate may be employed.

The solid tablet compositions may include a coating adapted to protect the composition from unwanted chemical changes, (e.g., chemical degradation prior to the release of the active drug substance). The coating may be applied on the solid dosage form in a similar manner as that described in Encyclopedia of Pharmaceutical Technology.

Controlled Release Oral Dosage Forms. Controlled release compositions for oral use may, e.g., be constructed to release the active drug by controlling the dissolution and/or the diffusion of the active drug substance. Illustrative sustained release formulations are described in U.S. Pat. Nos. 3,847,770; 3,916,899; 3,536,809; 3,598,123; 3,630,200; 4,008,719; 4,687,610; 4,769,027; 5,674,533; 5,059,595; 5,591,767; 5,120,548; 5,073,543; 5,639,476; 5,354,566; and 5,733,566, the disclosures of which are incorporated herein by reference.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated metylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

A controlled release composition containing one or more of the compounds of the claimed combinations may also be in the form of a buoyant tablet or capsule (i.e., a tablet or capsule that, upon oral administration, floats on top of the gastric content for a certain period of time). A buoyant tablet formulation of the compound(s) can be prepared by granulating a mixture of the drug(s) with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

Liquids for Oral Administration. Powders, dispersible powders, or granules suitable for preparation of an aqueous suspension by addition of water are convenient dosage forms for oral administration. Formulation as a suspension provides the active ingredient in a mixture with a dispersing or wetting agent, suspending agent, and one or more preservatives. Suitable dispersing or wetting agents are, for example, naturally-occurring phosphatides (e.g., lecithin or condensation products of ethylene oxide with a fatty acid, a long chain aliphatic alcohol, or a partial ester derived from fatty acids) and a hexitol or a hexitol anhydride (e.g., polyoxyethylene stearate, polyoxyethylene sorbitol monooleate, polyoxyethylene sorbitan monooleate, and the like). Suitable suspending agents are, for example, sodium carboxymethylcellulose, methylcellulose, sodium alginate, and the like.

Parenteral Compositions. The pharmaceutical composition may also be administered parenterally by injection, infusion or implantation (intravenous, intramuscular, subcutaneous, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy.

Compositions for parenteral use may be provided in unit dosage forms (e.g., in single-dose ampoules), or in vials containing several doses and in which a suitable preservative may be added (see below). The composition may be in form of a solution, a suspension, an emulsion, an infusion device, or a delivery device for implantation, or it may be presented as a dry powder to be reconstituted with water or another suitable vehicle before use. Apart from the active drug(s), the composition may include suitable parenterally acceptable carriers and/or excipients. The active drug(s) may be incorporated into microspheres, microcapsules, nanoparticles, liposomes, or the like for controlled release. Furthermore, the composition may include suspending, solubilizing, stabilizing, pH-adjusting agents, and/or dispersing agents.

As indicated above, the pharmaceutical compositions described herein may be in the form suitable for sterile injection. To prepare such a composition, the suitable active drug(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

Controlled Release Parenteral Compositions. Controlled release parenteral compositions may be in form of aqueous suspensions, microspheres, microcapsules, magnetic microspheres, oil solutions, oil suspensions, or emulsions. Alternatively, the active drug(s) may be incorporated in biocompatible carriers, liposomes, nanoparticles, implants, or infusion devices. Materials for use in the preparation of microspheres and/or microcapsules are, e.g., biodegradable/bioerodible polymers such as polygalactin, poly-(isobutyl cyanoacrylate), poly(2-hydroxyethyl-L-glutamnine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters)).

Rectal Compositions. For rectal application, suitable dosage forms for a composition include suppositories (emulsion or suspension type), and rectal gelatin capsules (solutions or suspensions). In a typical suppository formulation, the active drug(s) are combined with an appropriate pharmaceutically acceptable suppository base such as cocoa butter, esterified fatty acids, glycerinated gelatin, and various water-soluble or dispersible bases like polyethylene glycols and polvoxyethylene sorbitan fatty acid esters. Various additives, enhancers, or surfactants may be incorporated.

Compositions for Inhalation. For administration by inhalation, typical dosage forms include nasal sprays and aerosols. In a typically nasal formulation, the active ingredient(s) are dissolved or dispersed in a suitable vehicle. The pharmaceutically acceptable vehicles and excipients (as well as other pharmaceutically acceptable materials present in the composition such as diluents, enhancers, flavoring agents, and preservatives) are selected in accordance with conventional pharmaceutical practice in a manner understood by the persons skilled in the art of formulating pharmaceuticals.

Percutaneous and Topical Compositions. The pharmaceutical compositions may also be administered topically on the skin for percutaneous absorption in dosage forms or formulations containing conventionally non-toxic pharmaceutical acceptable carriers and excipients including microspheres and liposomes. The formulations include creams, ointments, lotions, liniments, gels, hydrogels, solutions, suspensions, sticks, sprays, pastes, plasters, and other kinds of transdermal drug delivery systems. The pharmaceutically acceptable carriers or excipients may include emulsifying agents, antioxidants, buffering agents, preservatives, humectants, penetration enhancers, chelating agents, gel-forming agents, ointment bases, perfumes, and skin protective agents.

The pharmaceutical compositions described above for topical administration on the skin may also be used in connection with topical administration onto or close to the part of the body that is to be treated. The compositions may be adapted for direct application or for introduction into relevant orifice(s) of the body (e.g., rectal, urethral, vaginal or oral orifices). The composition may be applied by means of special drug delivery devices such as dressings or alternatively plasters, pads, sponges, strips, or other forms of suitable flexible material.

Controlled Release Percutaneous and Topical Compositions. There are several approaches for providing rate control over the release and transdermal permeation of a drug, including: membrane-moderated systems, adhesive diffusion-controlled systems, matrix dispersion-type systems, and microreservoir systems. A controlled release percutaneous and/or topical composition may be obtained by using a suitable mixture of the above-mentioned approaches.

In a membrane-moderated system, the active drug is present in a reservoir which is totally encapsulated in a shallow compartment molded from a drug-impermeable laminate, such as a metallic plastic laminate, and a rate-controlling polymeric membrane such as a microporous or a non-porous polymeric membrane (e.g., ethylene-vinyl acetate copolymer). The active compound is only released through the rate-controlling polymeric membrane. In the drug reservoir, the active drug substance may either be dispersed in a solid polymer matrix or suspended in a viscous liquid medium such as silicone fluid. On the external surface of the polymeric membrane, a thin layer of an adhesive polymer is applied to achieve an intimate contact of the transdermal system with the skin surface. The adhesive polymer is preferably a hypoallergenic polymer that is compatible with the active drug.

In an adhesive diffusion-controlled system, a reservoir of the active drug is formed by directly dispersing the active drug in an adhesive polymer and then spreading the adhesive containing the active drug onto a flat sheet of substantially drug-impermeable metallic plastic backing to form a thin drug reservoir layer. A matrix dispersion-type system is characterized in that a reservoir of the active drug substance is formed by substantially homogeneously dispersing the active drug substance in a hydrophilic or lipophilic polymer matrix and then molding the drug-containing polymer into a disc with a substantially well-defined surface area and thickness. The adhesive polymer is spread along the circumference to form a strip of adhesive around the disc.

In a microreservoir system, the reservoir of the active substance is formed by first suspending the drug solids in an aqueous solution of water-soluble polymer, and then dispersing the drug suspension in a lipophilic polymer to form a plurality of microscopic spheres of drug reservoirs.

Dosages. The dosage of each compound of the claimed combinations depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect the dosage used.

As described above, the compound in question may be administered orally in the form of tablets, capsules, elixirs or syrups, or rectally in the form of suppositories. Parenteral administration of a compound is suitably performed, for example, in the form of saline solutions or with the compound incorporated into liposomes. In cases where the compound in itself is not sufficiently soluble to be dissolved, a solubilizer such as ethanol can be applied.

In addition to the foregoing illustrative dosages and dosing protocols, it is to be understood that an effective amount of any one or a mixture of the compounds described herein can be readily determined by the attending diagnostician or physician by the use of known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved, the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

Suitable routes for parenteral administration include intravenous, intraarterial, intraperitoneal, epidurial, intraurethral, intrasternal, intramuscular and subcutaneous, as well as any other art recognized route of parenteral administration. Suitable means of parenteral administration include needle (including microneedle) injectors, needle-free injectors and infusion techniques, as well as any other means of parenteral administration recognized in the art. Parenteral formulations are typically aqueous solutions which may contain excipients such as salts, carbohydrates and buffering agents (preferably at a pH in the range from about 3 to about 9), but, for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilization, may readily be accomplished using standard pharmaceutical techniques well known to those skilled in the art.

In addition, in those embodiments described herein drawn to combination therapy, such as compositions and/or methods that include one or more dimebolins and one or more NMDA antagonists, and/or pharmaceutically acceptable salts of the foregoing, the therapeutically effective amount refers to that amount of the combination of agents taken together so that the combined effect elicits the desired biological or medicinal response. For example, the therapeutically effective amount of one or more dimebolins and one or more NMDA antagonists would be the amount of the one or more dimebolins and the amount of the one or more NMDA antagonists that when taken together, contemporaneously, or sequentially have a combined effect that is therapeutically effective. Further, it is appreciated that in some embodiments of such methods that include co-administration, that coadministration amount of the one or more dimebolins or the one or more NMDA antagonists when taken individually may or may not be therapeutically effective.

It is also appreciated that the therapeutically effective amount, whether referring to monotherapy or combination therapy, is advantageously selected with reference to any toxicity, or other undesirable side effect, that might occur during administration of one or more of the compounds described herein. Further, it is appreciated that the co-therapies described herein may allow for the administration of lower doses of compounds that show such toxicity, or other undesirable side effect, where those lower doses are below thresholds of toxicity or lower in the therapeutic window than would otherwise be administered in the absence of a cotherapy.

In another illustrative embodiment, kits or packages are described herein. Illustrative kits and packages include preparations where the co-administered compounds are conveniently placed in a format following the dosing protocols described herein. For example, an illustrative package may include a grid pattern, wherein each section includes a dual or triple bubble pack for the one or more dimebolins and/or pharmaceutically acceptable salts thereof and illustratively the NMDA antagonist dosage, the stain dosage, the immunosuppressive drug dosage, and/or the immunomodulatory drug dosage. It is appreciated that other configurations that include the immunosuppressive drugs, the immunomodulatory drugs, or both, and the NMDA antagonist and the immunosuppressive or the immunomodulatory drug are described herein.

In another embodiment, methods are described herein for treating Primary Progressive Multiple Sclerosis that include the step of co-administering a therapeutically effective amount of one or more dimebolins and/or pharmaceutically acceptable salts thereof with one or more inhibitors of HMG-CoA reductase, also referred to as statins. In another embodiment, methods are described herein for treating Primary Progressive Multiple Sclerosis that include the step of co-administering a therapeutically effective amount of one or more dimebolins and/or pharmaceutically acceptable salts thereof with one or more immunomodulatory drugs. In another embodiment, methods are described herein for treating Primary Progressive Multiple Sclerosis that include the step of co-administering a therapeutically effective amount of one or more dimebolins and/or pharmaceutically acceptable salts thereof with one or more immunosuppressive drugs.

In another embodiment, methods are described herein for treating Secondary Progressive Multiple Sclerosis that include the step of co-administering a therapeutically effective amount of one or more dimebolins and/or pharmaceutically acceptable salts thereof with one or more inhibitors of HMG-CoA reductase, also referred to as statins. In another embodiment, methods are described herein for treating Secondary Progressive Multiple Sclerosis that include the step of co-administering a therapeutically effective amount of one or more dimebolins and/or pharmaceutically acceptable salts thereof with one or more immunomodulatory drugs. In another embodiment, methods are described herein for treating Secondary Progressive Multiple Sclerosis that include the step of co-administering a therapeutically effective amount of one or more dimebolins and/or pharmaceutically acceptable salts thereof with one or more immunosuppressive drugs.

Examples of illustrative methods of administration include, but are not limited to, oral (po), intravenous (iv), intramuscular (im), subcutaneous (sc), transdermal, and rectal. Compounds may also be administered directly to the nervous system including, but not limited to, intracerebral, intraventricular, intracerebroventricular, intrathecal, intracisternal, intraspinal and/or peri-spinal routes of administration by delivery via intracranial or intravertebral needles and/or catheters with or without pump devices.

In another embodiment, the methods described herein include the use of controlled release and/or slow release formulations of the compounds and/or combination of compounds described herein are described. It is appreciated that a controlled release and/or slow release formulation of one or more dimebolins and/or pharmaceutically acceptable salts thereof may be advantageous for maintaining therapeutically effective blood levels in between doses. In another embodiment, formulations suitable for parenteral administration are described herein, including formulations suitable for pumps and or patches that may be adhered to or worn by a patient.

It is to be understood that a wide range of doses of one or more dimebolins or pharmaceutically acceptable salts thereof, either alone or in combination with another component, may be used in the methods and compositions described herein. In addition, any suitable route of administration may be used in the methods described herein. In addition, any suitable formulation may be used for the compositions described herein.

In another illustrative embodiment, oral formulations of one or more dimebolins or pharmaceutically acceptable salts thereof, either alone or in combination with another component, are described. In another embodiment, the methods described herein are adapted for treating progressive MS, including primary progressive and secondary progressive MS. The methods include to step of administering an oral formulation of one or more dimebolins, or pharmaceutically acceptable salts thereof, to a patient suffering from or in need of relief from the disease. Without being bound by theory, it is believed herein that oral administration and oral formulations may be particularly applicable to treating progressive forms of MS.

In another illustrative embodiment, parenteral formulations of one or more dimebolins or pharmaceutically acceptable salts thereof, either alone or in combination with another component, are described. In another embodiment, the methods described herein are adapted for treating relapsing MS, including relapsing and relapsing-remitting MS. The methods include to step of administering a parenterial formulation of one or more dimebolins, or pharmaceutically acceptable salts thereof, to a patient suffering from or in need of relief from the disease. Without being bound by theory, it is believed herein that parenteral administration and parenteral formulations may be particularly applicable to treating relapsing forms of MS.

In another illustrative embodiment of the methods described herein, a compound described herein is administered to a patient orally in the range from about 0.01 mg/kg to about 100 mg/kg, or illustratively in the range from about 0.01 mg/kg to about 10 mg/kg, or from about 0.1 mg/kg to about 10 mg/kg, or illustratively in the range from about 0.01 mg/kg to about 1 mg/kg, or about 0.1 mg/kg to about 1 mg/kg. In another illustrative embodiment of the methods described herein, a compound described herein is administered to a patient parenterally in the range from about 0.01 mg/kg to about 100 mg/kg, or illustratively in the range from about 0.01 mg/kg to about 10 mg/kg, or from about 0.1 mg/kg to about 10 mg/kg, or illustratively in the range from about 0.01 mg/kg to about 1 mg/kg, or about 0.1 mg/kg to about 1 mg/kg. Each of the foregoing may be illustratively administered q.d., b.i.d., and/or t.i.d. Accordingly, the equivalent daily dose for adults is in the range from about 10 mg to about 1 g per day.

Illustratively for an adult, the dimebolin or pharmaceutically acceptable salt is administered orally 20 mg t.i.d. for a total daily dose of 60 mg. Alternatively, the dimebolin or pharmaceutically acceptable salt is administered orally 10 mg t.i.d. for a total daily dose of 30 mg, or 40 mg t.i.d. for a total daily dose of 120 mg, or 60 mg t.i.d. for a total daily dose of 180 mg, or 80 mg t.i.d. for a total daily dose of 240 mg. Illustratively, the dimebolin or pharmaceutically acceptable salt is administered parenterally 10 mg t.i.d. for a total daily dose of 30 mg. Alternatively, the dimebolin or pharmaceutically acceptable salt is administered orally 5 mg t.i.d. for a total daily dose of 15 mg, or 20 mg t.i.d. for a total daily dose of 60 mg.

In another embodiment, the methods described herein include the use of tablets for pediatric use that include 5 mg b.i.d. or t.i.d. of dimebolin and/or one or more analogs or derivatives thereof. Additional illustrative pediatric doses are 0.5 to 7.5 mg/day (under 1 year of age), 5 to 15 mg/day (1 to 2 years of age), 7.5 to 30 mg/day (from 3 to 5 years of age), 20-40 mg/day (over 5 years of age), each administered b.i.d. or t.i.d.

In another embodiment the methods described herein include a titration step where the dose is gradually increased over a predetermined time period, such as a two step protocol for adults as follows: 10 mg thrice daily for 7 days, then 20 mg thrice daily.

In another embodiment, the methods described herein include a titration step where the dose is gradually increased over a predetermined time period, such as a two step protocol for pediatric patients as follows: 2.5 mg thrice daily for 7 days, then 5 mg thrice daily, 5 mg thrice daily for 7 days, then 10 mg thrice daily.

Optimal dosages and dosage regimens to be administered may be readily determined by routine experimentation, and it is understood that such optimal dosages and dosage regimens will vary with the mode of administration, the strength of the preparation and the advancement of the disease condition. In addition, factors associated with the particular patient being treated, including patient's sex, age, weight, diet, physical activity, time of administration and concomitant diseases, will result in the need to adjust dosages and/or regimens.

Without limiting the foregoing, it is appreciated that such lower doses of dimebolins may be more applicable to an ongoing, or chronic therapy, designed for continuous administration, rather than intermittent or acute administration. Accordingly, the daily dose may be divided and administered b.i.d. and/or t.i.d, although it is to be understood that q.d. dosing is described herein. It is to be understood that the illustrative doses described herein represent daily doses, and may be therefore administered q.d., b.i.d., t.i.d., and according to additional dosing protocols. In addition, it is to be understood that the doses may be single or divided.

In one variation of each of the foregoing embodiments, the one or more dimebolins or pharmaceutically acceptable salts thereof, are administered t.i.d.

In another embodiment, pharmaceutical composition packages are described herein. In one embodiment, the package includes a therapeutically effective amount of one or more dimebolins and a therapeutically effective amount of one or more NMDA receptor antagonists, or pharmaceutically acceptable salts of the foregoing, each adapted for co-administration. In another embodiment, the package includes a therapeutically effective amount of one or more dimebolins and a therapeutically effective amount of one or more statins, or pharmaceutically acceptable salts of the foregoing; each adapted for co-administration. In another embodiment, the package includes a therapeutically effective amount of one or more dimebolins, a therapeutically effective amount of one or more NMDA receptor antagonists, and a therapeutically effective amount of one or more immunomodulatory drugs, or pharmaceutically acceptable salts of the foregoing, each adapted for co-administration.

It is to be understood that in each of the foregoing embodiments of the methods and medicaments described herein, any one or more of the dimebolins described herein may be included. For example, in each of the foregoing embodiments of the methods and medicaments described herein, the dimebolin may be Dimebon (as the hydrochloride), or other pharmaceutically acceptable salt thereof.

The effective use of the methods described herein for treating or ameliorating MS using one or more compounds described herein may be based upon animal models, such as murine and rabbit models. For example, it is understood that MS in humans are characterized by a loss of function, and/or the development of symptoms, each of which may be elicited in animals, such as mice and rabbits, and other surrogate test animals. Illustrative animal models of Multiple Sclerosis that may be used to evaluate the methods of treatment and the pharmaceutical compositions described herein to determine the therapeutically effective amounts described herein, include but not limited to the mouse SJL, NOD, and C57BL/6J mice (Taconic or The Jackson Laboratory), in which EAE is induced by subcutaneous immunization of 10-week-old NOD mice or 7-week-old C57BL/6J into the flanks with 200 μl of an emulsion containing 150 μg of MOG35-55 peptide (MEVGWYRSPFSRVVHLYRNGK; David Teplow, David Geffen School of Medicine, UCLA, Los Angeles, Calif., USA) and 400 μg of Mycobacterium tuberculosis extract H37 Ra (Difco) in incomplete Freund's adjuvant oil. In addition, the animals receive 150 ng of pertussis toxin (List Biological Laboratories) i.p. on day 0 and day 2. EAE in SJL mice is induced by immunization with 50 μg of PLP131-151 peptide in complete Freund's adjuvant oil.

The following examples further illustrate specific embodiments of the invention; however, the following illustrative examples should not be interpreted in any way to limit the invention.

### EXAMPLES

### Example

Dimebon (20 mg tablet, oral) is administered three times daily (60 mg daily dose) to a patient suffering from or in need of relief from Multiple Sclerosis. Illustratively, the dimebon in the form of tablets (comprising 20 mg of dimebon, 30 mg of lactose, and 5 mg of magnesium stearate) for oral administration. The duration of treatment in this and other examples described herein is determined according to the progression of Multiple Sclerosis in each individual patient and dose adjustments are made accordingly. Treatment efficacy in this and other examples described herein is monitored by self-reporting and the results of treatment are evaluated statistically using Student's t-test and/or Fisher's “Fi” criterion.

### Example

Alternative non-limiting examples include adult doses of 10 mg (½ tablet, oral) tid, 20 mg (1 tablet, oral) tid, 30 mg (1 and ½ tablet, oral) tid, 40 mg (2 tablets, oral) tid, 10 mg (½ tablet, oral) bid, 20 mg (1 tablet, oral) bid, 30 mg (1 and ½ tablet, oral) bid, and 40 mg (2 tablets, oral) bid.

### Example

Alternative non-limiting titration examples include adult dose titrations according to the following protocols: 5 mg (pediatric tablet, oral) tid for first week, 10 mg (½ tablet, oral) tid for second week, and 20 mg (1 tablets, oral) tid for third week and successive treatment, and 10 mg (½ tablet, oral) tid for first week, and 20 mg (1 tablets, oral) tid for second week and successive treatment.

It is to be understood herein that additional titration protocols are described herein by the appropriate selection of pediatric and adult doses, including ½ tablet doses such as 10 mg adult half tablets and 2.5 mg pediatric half tablets.

### Example

Dimebon (5 mg tablet, oral) is administered three times daily to a pediatric patient suffering from or in need of relief from Multiple Sclerosis Illustratively, the dimebon in the form of tablets (comprising 5 mg of dimebon, 10-20 mg of lactose, and 1-5 mg of magnesium stearate) for oral administration. The duration of treatment in this and other examples described herein is determined according to the progression of Multiple Sclerosis in each individual patient and dose adjustments are made accordingly. Treatment efficacy in this and other examples described herein is monitored by self-reporting and the results of treatment are evaluated statistically using Student's t-test and/or Fisher's “Fi” criterion.

### Example

Alternative non-limiting examples include pediatric doses of 2.5 mg (½ tablet, oral) tid, 5 mg (1 tablet, oral) tid, 7.5 mg (1 and ½ tablet, oral) tid, 10 mg (2 tablets, oral) tid, 2.5 mg (½ tablet, oral) bid, 5 mg (1 tablet, oral) bid, 7.5 mg (1 and ½ tablet, oral) bid, and 10 mg (2 tablets, oral) bid.

### Example

Dimebon is administered as described herein, such as oral administration of 20 mg, three times daily, to a patient diagnosed with secondary progressive multiple sclerosis, and co-administered with 10 mg oral memantine b.i.d. In one variation, one or more of dimebon and/or memantine may be titrated in as described above. For example, memantine may be started with 5 mg daily of (half a tablet in the morning) during the 1st week. In the 2nd week 10 mg per day (half a tablet twice a day) and in the 3rd week 15 mg per day (one tablet in the morning and half a tablet in the afternoon or evening) is recommended. From the 4th week on, treatment can be continued with the recommended maintenance dose of 20 mg per day (one tablet twice a day).

### Example

Dimebon is administered as described herein, such as oral administration of 20 mg, three times daily, and co-administered with oral simvastatin (20 mg tablet, Merck & Co Inc) once daily.

### Example

Dimebon is administered as described herein, such as oral administration of 20 mg, three times daily, and co-administered with oral simvastatin (10 mg tablet, Merck & Co Inc) once daily.

### Example

Dimebon is administered as described herein, such as oral administration of 20 mg, three times daily, and co-administered with oral simvastatin (40 mg tablet, Merck & Co Inc) once daily.

### Example

Dimebon is administered as described herein, such as oral administration of 20 mg, three times daily, and co-administered with oral simvastatin (80 mg tablet, Merck & Co Inc) once daily.

### Example

In each of the foregoing Examples of coadministration of dimebolin with simvastatin, it is to be understood herein that additional examples are described where simvastatin is replaced with lovastatin at the same indicated dose, e.g. 10 mg, 20 mg, 40 mg, or 80 mg q.d.

### Example

Dimebon is administered as described herein, such as oral administration of 20 mg, three times daily, and co-administered with glatiramer acetate 20 mg subcutaneously (Teva) one time daily.

### Example

Dimebon is administered as described herein, such as oral administration of 20 mg, three times daily, to a patient diagnosed with primary progressive or secondary progressive multiple sclerosis, and co-administered with oral simvastatin (10-80 mg, such as 20 mg oral tablet) q.d., and co-administered with glatiramer acetate (20 mg sc inj, Teva) one time daily.

### Example

Dimebon is administered as described herein, such as oral administration of 20 mg, three times daily, to a patient diagnosed with primary progressive or secondary progressive multiple sclerosis, and co-administered with oral azathioprine (50-mg scored Tablets 100 mg (as the sodium salt) for I.V. injection, GlaxoSmithKline) two times daily.

### Example

The previous example may include a titration protocol for azathioprine as follows: The initial dose should be approximately 1.0 mg/kg (50 to 100 mg) given as a single dose or on a twice-daily schedule. The dose may be increased, beginning at 6 to 8 weeks and thereafter by steps at 4-week intervals,

### Example

Dimebon is administered as described herein, such as oral administration of 20 mg, three times daily, to a patient diagnosed with relapsing-remitting multiple sclerosis, and co-administered with oral mycophenolate-mofetil (500 mg tablet, 250 mg capsule, Roche) two times daily. A dose of 1 g administered orally or intravenously (over NO LESS THAN 2 HOURS) twice a day (daily dose of 2 g) is recommended for use in renal transplant patients. Although a dose of 1.5 g administered twice daily (daily dose of 3 g) was used in clinical trials and was shown to be safe and effective, no efficacy advantage could be established for renal transplant patients. Patients receiving 2 g/day of CellCept demonstrated an overall better safety profile than did patients receiving 3 g/day of CellCept.

### Example

Dimebon is administered as described herein, such as oral administration of 20 mg, three times daily, to a patient diagnosed with progressive-relapsing multiple sclerosis, and co-administered with oral mycophenolate-mofetil (500 mg tablet, Roche) two times daily.

### Example

Determination of permeability transition in permeabilized cells. Materials and products. Cells from an oral squamous carcinoma cell line, such as KB cells, are maintained in exponential growth phase using RPMI 1640 culture medium, supplemented with 10% (v/v) fetal calf serum, 2 mM glutamine, 50 units/ml penicillin and 50 μg/ml streptomycin. These cells may be purchased from A.T.C.C. (reference CCL-17). Calcein-acetomethoxyl ester and Calcium Green-5N are obtained from Molecular Probes; monoclonal antibodies are from BD Biosciences Pharmingen (San Diego, Calif., U.S.A.). All other chemicals may be purchased from commercial suppliers.

Intact KB cells (5×106) are incubated for 30 min without (control) or with a dimebolin such as Dimebon, at appropriate concentrations, such as at 25 μM, or in the range from about 25 μM to about 10 mM. The cells are then centrifuged and resuspended in a medium containing 250 mM sucrose, 10 mM Mops, 1 mM Pi/Tris and 50 μg/ml digitonin (pH 7.35) and placed in a spectrofluorimeter glass cuvette, continuously stirred and thermostatically maintained at 25° C. After 2 min, cells are permeabilized and 1 μM CsA or vehicle is also added to the medium as indicated. After signal stabilization, 10 μl of 1 mM Ca2+ pulses is successively added at 2 min intervals until the opening of PTP, as indicated by the release of Ca2+ in the medium. Measurements of Ca2+ are performed fluorimetrically with a PTI Quantamaster C61 spectrofluorimeter. Free Ca2+ is measured in the presence of 0.25 μM Calcium Green-5N with excitation and emission wavelengths set at 506 and 532 nm respectively.

Determination of permeability transition in intact cells. Calcein staining in KB cells is achieved after the cells (5×104) are grown for 48 h on 22-mm diameter round glass coverslips and exposed for 15 min at 37° C. to a PBS medium supplemented with 5 mM glucose, 0.35 mM pyruvate, 1 mM CoCl₂ and 1 μM calcein-acetomethoxyl ester. After loading, cells are washed free of calcein and CoCl₂ and further incubated for 20 min at 37° C. in PBS/glucose/pyruvate medium, supplemented with either 1 μM CsA or vehicle, or with a dimebolin, such as dimebon, such as at 25 μM, or in the range from about 25 μM to about 10 mM. For low concentrations of the dimebolin, KB cells are first preincubated for 24 h without (control) or with a dimebolin, such as dimebon, at appropriate concentrations, such as at 10 μM, or in the range from about 10 μM to about 100 μM, or in the range from about 10 μM to about 1 mM, before the calcein and CoCl₂ loading step. Coverslips are then mounted on the stage of an inverted microscope and PTP opening is achieved by adding 50 μM tBH (t-butyl hydroperoxide), a glutathione-oxidizing agent. Changes in cellular fluorescence are quantified using an appropriate imaging software. The intensity of fluorescence of ten cells is followed in time after the addition of tBH.

Determination of cellular death. KB cells (2×107) are preincubated in Petri dishes with either 1 μM CsA or vehicle, or with a dimebolin, such as dimebon, at appropriate concentrations, such as at 25 μM, or in the range from about 25 μM to about 10 mM, for 30 min, or preincubated for 24 h at 37° C. without (control) or with the dimebolin, at appropriate concentrations, such as at 10 μM, or in the range from about 10 μM to about 100 μM, or in the range from about 10 μM to about 1 mM. Cells are washed with PBS before subsequent exposure to 0.2 mM tBH for 45 min. Cells are again washed with PBS and incubated at 37° C. for 6 or 24 h in a complete RPMI 1640 medium. Cytotoxicity is evaluated either by staining necrotic cells with 20 μg/ml propidium iodide or by using a Trypan Blue (5%, v/v) exclusion assay.

Cellular images are obtained at 25° C., such as with a Nikon TE200 microscope (Nikon France, Champigny-sur-Marne, France), which is equipped for epifluorescent illumination and includes a xenon light source (75 W) and a 12-bit digital-cooled charge-coupled-device camera (SPOT-RT; Diagnostic Instruments, Sterling Heights, Mich., USA). For calcein fluorescence, 488±5/525±10 nm excitation/emission filter settings are used, and images are collected every minute with a constant exposure time using a 60×/1.40 Plan Apo oil immersion objective (Nikon). For detection of propidium iodide, five randomly selected fields are acquired from each Petri dish using an excitation/emission cube of 550±10/580 longpass and an ELWD 20×/0.45 Plan Fluor objective (Nikon). The corresponding bright field images are also obtained, and the two channels are overlaid using appropriate software, such as the appropriate function of the SPOT 3.0.6 software.

Cytochrome c is assessed in both mitochondrial and cytosolic spaces after KB cells are fractionated using the digitonin method. Cytosolic (3 μg) and mitochondrial proteins (15 μg) are separated by SDS/PAGE (10% gel) in Mes buffer, followed by Western-blot analysis. Membranes are probed with a monoclonal antibody against cytochrome c (1 μg/ml) clone 7H8.2C12, and developed with a secondary goat anti-mouse horseradish peroxidase-labelled antibody, followed by chemiluminescent detection. Quantification is performed using an appropriate imaging software.

Statistics. Results may be expressed as means±S.E.M. and statistically significant differences may be assessed by ANOVA, followed by Fisher's PLSD (protected least-significant difference) post hoc test or by paired or unpaired Student's t test.

### Example

One or more dimebolins and/or pharmaceutically acceptable salts thereof, including the compounds described herein, are shown to be efficacious in the mouse MOG-induced model of chronic progressive EAE. Briefly, chronic progressive EAE in SJL mice is induced by immunization with 50 μg of PLP131-151 peptide in complete Freund's adjuvant. Animals are kept in a conventional pathogen-free facility. For example, Dimebon is given daily beginning on day 20 at 10 mg/kg intravenously. Vehicle consisting of 2% DMSO. Clinical signs of EAE are assessed according to the following score: 0, no signs of disease; 1, loss of tone in the tail; 2, hindlimb paresis; 3, hindlimb paralysis; 4, tetraplegia; 5, moribund. (Forte M, Gold B G, Marracci G, Chaudhary P, Basso E, Johnsen D, Yu X, Fowlkes J, Rander M, Stem K, Bernardi P, Bourdette D. Cyclophilin D inactivation protects axons in experimental autoimmune encephalomyelitis, an animal model of multiple sclerosis. Proc Natl Acad Sci USA. 2007 May 1; 104(18):7558-63. Epub 2007 Apr. 26.)

### Example

Severe EAE Animal Model of MS. Experimental Autoimmune Encephalomyelitis (EAE), also called Experimental Allergic Encephalomyelitis, can be induced in healthy animals, such as mice, rats, guinea pigs, rabbits, macaques, rhesus monkeys and marmosets, by injecting whole or parts of various proteins that make up myelin, such as Myelin Basic Protein (MBP), Proteolipid Protein (PLP), and Myelin Oligodendrocyte Glycoprotein (MOG). Briefly, to generate severe EAE, 12-week-old C57BL/6 female mice (Jackson Laboratory, Bar Harbor, Me., USA) are injected subcutaneously at the back of the tail and 7 days later in the flanks with 300 μg of MOG35-55 peptide (Bernard C C, Johns T G, Slavin A J, Ichikawa M, Ewing C, Liu J, et al. Myelin oligodendrocyte glycoprotein: a novel candidate autoantigen in multiple sclerosis. [Review]. J Mol Med 1997; 75: 77-88.). emulsified in 100 μl of complete Freund's adjuvant (CFA) (Difco Laboratories, Detroit, Mich., USA) containing an additional 4 mg/ml of Mycobacterium tuberculosis (H37Ra). Mice are injected intraperitoneally with 300 ng of reconstituted lyophilized pertussis toxin (List Biological Laboratories, Campbell, Calif., USA) in 200 μl of PBS. The pertussis toxin injection is repeated after 48 h (Liu J, Marino M W, Wong G, Grail D, Dunn A, Bettadapura J, et al. TNF is a potent anti-inflammatory cytokine in autoimmune-mediated demyelination. Nat Med 1998; 4: 78-83).

### Example

Mild EAE Animal Model of MS. Briefly, to obtain mild EAE, only one injection of MOG35-55 peptide emulsified in 100 μl of CFA is given to 12-week-old C57BL/6 female mice (Jackson Laboratory, Bar Harbor, Me., USA). Subsequently, the dose is reduced from 300 to 25 μg, and M. tuberculosis is not supplemented to the CFA. Pertussis toxin is continued to be administered as with the severe EAE regimen.

### Example

Equivalent models of MS using SJL/J Mouse (Jackson Laboratories) EAE and Dark Agouti (DA) Rat (Harlan) EAE are also described. Details of the models are described in U.S. Pat. No. 7,041,661, the disclosure of which is incorporated herein by reference.

### Example

Evaluation of Dimebolins and Pharmaceutically Acceptable Salts Thereof. Animals are treated intraperitoneally daily, beginning on the day of MOG induction, with a dimebolin or vehicle (saline). Seven to nine animals per group are used. Control EAE mice are injected with 200 μl saline daily, while the dimebolin EAE mice receive the dimebolin in the range from about 1 mg/kg to about 50 mg/kg once or twice a day, from 1 mg/kg to about 25 mg/kg once or twice a day, or about 1 mg/kg once or twice a day until they are sacrificed. In a last group of mice, treatment with intraperitoneal dimebon (in the range from about 5 mg/kg to about 50 mg/kg daily, from 5 mg/kg to about 25 mg/kg daily, or about 5 mg/kg 25 mg/kg daily) is initiated only from Day 10 post-immunization, when clinical disease is beginning to be apparent in mice.

All mice are weighed on a daily basis. Severity of EAE is graded according to Liu J, Marino M W, Wong G, Grail D, Dunn A, Bettadapura J, et al. TNF is a potent anti-inflammatory cytokine in autoimmune-mediated demyelination. Nat Med 1998; 4: 78-83). Grades are: 0, no disease; 1, limp tail; 2, partial paralysis of one or two hind limbs; 3, complete paralysis of hind limbs; 4, hind limb paralysis and fore limb paraparesis; 5, moribund.

For histological analyses, anaesthetized mice are perfused with 40 ml of cold saline and the CNS is dissected. The sacral part of the spinal cord is immersed in 4% paraformaldehyde overnight and embedded in paraffin wax, cross sectioned at 6-8 μm and stained with Luxol fast blue for evidence of demyelination, and optionally stained with haematoxylin-eosin for evidence of inflammation. Optic nerves are fixed in 2.5% glutaraldehyde, embedded in epon, sectioned at 2 μm, and stained with toluidine blue.

The five vehicle-treated EAE mice, and the dimebolin-treated mice #1 and #2 are sacrificed at Days 19-21 after MOG immunization. Dimebon-treated mice #3 to #5 are killed at Day 27 when disease is full-blown. The anterior (Ant), lateral (Lat) and posterior (Post) columns of the right and left cord are evaluated blind, using a scale from 0 (no inflammation) to 3 (severe inflammation deep in the CNS parenchyma and which approaches grey matter areas).

### Example

Acute EAE Murine Model. Experimental Allergic Encephalomyelitis (EAE) is a central nervous system (CNS) autoimmune demyelinating disease that mimics many of the clinical and pathologic features of Multiple Sclerosis (MS). The MOG murine model consists of a sensitization period, induced by the single subcutaneous (SC) injection of MOG emulsified in Complete Freund's Adjuvant (CFA) on study days 0 and 6, followed by intraperitoneal (IP) supplemental immunostimulation with Pertussis Toxin (PT) carried out once at the time of EAE induction and once again 48 hours later. Additional details are described in Gilgun-Sherki Y. et al., Neurosciences Research 47:201-207, 2003.

Briefly, the study is performed in female C57BL/6J mice (Harlan Laboratories Israel, Ltd.) as young adults, 8-9 weeks of age at study initiation. animals are acclimatized to laboratory conditions at least 5 days. During acclimation and throughout the entire study duration, animals are housed in groups of 10 mice maximum in polypropylene cages, which are fitted with solid bottoms and filled with wood shavings as bedding material. Animals are provided ad libitum a commercial rodent diet and free access to drinking water, supplied to each cage via polyethylene bottles with stainless steel sipper tubes. Water is monitored periodically. Automatically controlled environmental conditions are set to maintain temperature at 20-24° C. with a relative humidity (RH) of 30-70%, a 12:12 hour light: dark cycle and 15-30 air changes/hr in the study room. Temperature and RH are monitored daily. The light cycle is monitored by the control clock. Weight variation of animals at the time of treatment initiation should not exceed ±20% of the mean weight. At the end of the study, surviving animals are euthanized by CO₂ asphyxiation.

Antigen. The MOG peptide (35-55 human, [H]-MEVGWYRPFSRVVHLYRNGK-[OH]) solution is freshly prepared prior to each inoculation session by dissolving the RP-HPLC-purified lyophilized powder in Phosphate Buffered Saline (PBS) to achieve a solution at a final injected concentration of 2 mg/ml. This concentration is appropriate for the selected dose and dose volume of 200 μg MOG in 100 μL PBS.

Sensitizer. Complete Freund's Adjuvant (CFA) suspension in mineral oil containing heat killed Mycobacterium Tuberculosis H37 Ra at a concentration of 3 mg/mL is used as supplied.

MOG/CFA Emulsion. Prior to each inoculation carried out on study days 0 and 6, 100 μl of MOG solution (200 μg) is emulsified with 100 μl of CFA suspension (300 μg heat killed Mycobacterium Tuberculosis H37 Ra). The solution is thoroughly mixed by employing two syringes connected by a Luer fitting to equal a total dose volume of 200 μl/animal.

Immunostimulant. Prior to the first immunostimulation injection performed on study day 0, the Pertussis Toxin (PT) stock solution (Clear liquid exotoxin, produced by Bordetella Pertussis, 0.2 mg/ml, Sigma-Aldrich) is freshly prepared by diluting the commercial PT sample (0.2 mg/mL, total of 50 μg/250 μL/vial) in sterile distilled water (Water for Injection) to achieve a stock concentration of 100 μg/mL.

Prior to each injection on study days 0 and 2, the PT stock solution (100 μg/mL) is diluted in Phosphate Buffered Saline (PBS) to achieve a final injected concentration of 2 μg/ml, which is appropriate for the selected dose level and volume dosage.

Following each PT injection, the daily vial is discarded. Diluted stock solution (100 μg/mL) is kept refrigerated (2-8° C.) and is reused for the second PT injection on study day 2. The solution is not used if more than 48 hours have passed following its preparation. Thorough vortexing is required just prior to each PT injection session.

All animals are subjected to a single inoculum injection of MOG/CFA on study days 0 (study commencement) and 6. The inoculum injection consists of a homogenate emulsive mixture of MOG and CFA as follows, MOG/CFA encephalitogenic emulsive inoculum (200 μg MOG/300 μg CFA) is injected at a total dose volume of 200 μL/animal and is delivered as 2×100 μL subcutaneous (SC) bilateral injections over the paralumbar regions.

In order to increase the permeability of the blood-brain barrier (BBB), all animals are subjected to supplemental immunostimulation by Pertussis Toxin (PT), which is administered at a dose level of 20 μg/kg (approximately 400 ng/mouse), by intraperitoneal (IP) injections at 2 occasions: once at the time of EAE induction on study day 0 and once 48 hours later (study day 2). Pertussis Toxin solution is injected at a volume dosage of 10 mL/kg.

### Example

Dimebon. The test compounds are made as a weekly concentrated stock solution, aliquoted in 7 vials and frozen at −20° C. One vial is thawed for daily administration. Aliquot preparation is performed once every week. Dexamethasone (positive control, Sigma), is diluted in ethanol to achieve a concentration of 1 mg/mL and diluted with distilled water to achieve the dose concentration of 0.05 mg/mL. Vehicle, test compounds, and dexamethasone positive control are administered IP once daily beginning on study day 0.

TABLE
Treatment Groups

| Group | Group | Test | Dose Level | Volume dosage |
|---|---|---|---|---|
| Number | size | Material | (mg/kg/admin) | (mL/kg) |
| 1F | n = 10 | Vehicle Control | 0 | 10 |
| 2F | n = 10 | Positive Control | 1.0 | 10 |
| | | (Dexamethasone) | | |
| 3F | n = 10 | Dimebon | 1.0 | 10 |
| 5F | n = 10 | Dimebon | 10.0 | 10 |

Throughout the 35-day study, clinical examinations are performed and recorded at least once daily in addition to the EAE clinical scoring and assessment. Observations include changes in skin, fur, eyes, mucous membranes, occurrence of secretions and excretions (e.g. diarrhea), autonomic activity (e.g. lacrimation, salivation, piloerection, pupil size, unusual respiratory pattern), gait, posture, and response to handling, as well as the presence of unusual behavior, tremors, convulsions, sleep, and coma.

Body weight loss can be the first sign of disease initiation, while a sudden marked weight gain tends to accompany remission of EAE symptoms. Therefore, determination of individual body weights of animals is made shortly before EAE induction on study day 0 (study commencement) and is monitored on a daily basis throughout the 35-day observation period.

Initially, all animals are examined for signs of any neurological responses and symptoms prior to EAE induction (study day 0) and are examined on a daily basis throughout the 35-day observation period. EAE reactions are scored and recorded according to a 0-15 scale Clinical signs Scoring Methods. The clinical Score is determined by summing the score of each section (Weaver at al, 2005).

| | Signs/Symptoms | Grade |
|---|---|---|
| Tail | none | 0 |
| | half paralyzed tail | 1 |
| | fully paralyzed tail | 2 |
| Limb (each hind and forelimb) | none | 0 |
| | weak or altered gait | 1 |
| | paresis | 2 |
| | fully paralyzed limb | 3 |
| Mortality | | 15 |

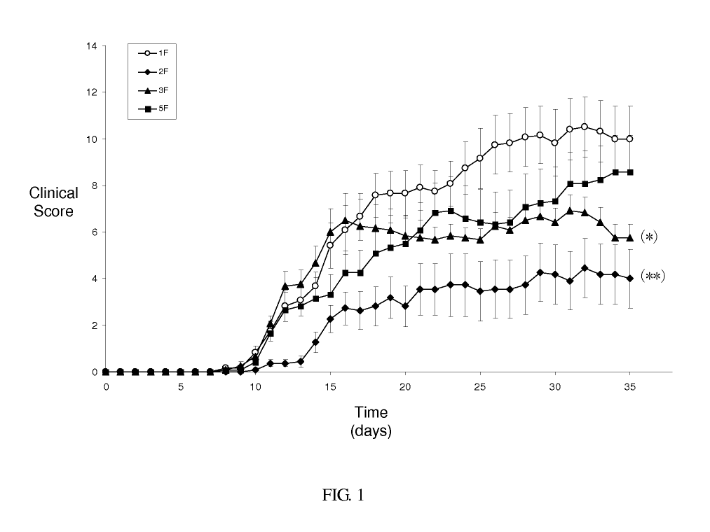

The results are shown in the FIG. 1.
The activity observed for the positive control (2F) was statistically significant compared to the vehicle-only treatment group (1F), p<0.01. The activity observed for the treatment group 3F was statistically significant compared to the vehicle-only treatment group (1F), p<0.05. Though the activity observed for the treatment group 5F was not statistically significant compared to the vehicle-only treatment group (1F) at the end of the study, p˜0.5, the intermediate time points on days 26 and 27 were statistically significant compared to the vehicle-only treatment group (1F), p<0.1.

1.-30. (canceled)

31. A method for treating multiple sclerosis in a patient in need thereof, the method comprising the step of administering to the patient a therapeutically effective amount of one or more dimebolins, or a pharmaceutically acceptable salt thereof.
